SEQUENCE LISTING (20 sequences)

SEQ ID NO: 1 - DNA, 122 bp, Homo sapiens
tcgcaaacaaagaagtcagttgggcgccccgaagctgctggccttctgagtggcggaaggcgccaggctcggtgattgaaaccacatgtgctggaggtttcaagagcagcgacccagacacc

SEQ ID NO: 2 - DNA, 122 bp, Homo sapiens
cgccagcttggtggccaaggtctgtgttgggggccactctctatccccccgactacaggtcgggagctgctggtgatggaaccaggggctgagtatgcagtgctggccacatactaggagaa

SEQ ID NO: 3 - DNA, 122 bp, Homo sapiens
aacaaatgttgcaacctggacatgaaaagcagtctgtctacccaaggctgcccccgcagccggatggcagcgccactccctcccccacaaccggggaacaaggaccagggccgggtggtgga

SEQ ID NO: 4 - DNA, 122 bp, Homo sapiens
cccatgctttttgcatggggaaaaaaggagcctggaccctctgccccataaactccataacgaactaaatatatgcacagttgtgctttgtgagttcaattagaattttggacaaggtttta

SEQ ID NO: 5 - DNA, 122 bp, Homo sapiens
tggggttcaggacggtgccagcatgcccagcaatcccggagttccccgtgctcaggaactcgcaccgctggctccacgctctctactccagtgggacaagaaagctctgctcaccaggcaac

SEQ ID NO: 6 - DNA, 122 bp, Homo sapiens
aataccagactaacaagtttggcctttatcttcgaaataatgggcaaccatagaagaaatcggagcatgtgagggtcaagataaaattttagttttggaattttccctctggtattgcgtgt

SEQ ID NO: 7 - DNA, 122 bp, Homo sapiens
tttcctccttttctttccttcccttccttcctccctctccccttcccccaccccccgccccgccccgccccagtcttggtggcttgttccggatctggtgttgctcattcactcatcaaaca

SEQ ID NO: 8 - DNA, 122 bp, Homo sapiens
agctgcacggagctataggtgctctgaagtcagacagtgcagccaagccagtgccttcgccgtccccattgaggaaggagagcctcaacattatttttaaacatacagaaaagttgttttcc

SEQ ID NO: 9 - DNA, 122 bp, Homo sapiens
tgccattggcagaacaaagcagtctcggcctggcttggcagcatccaaaaggaccgcagccgccctgccgctgcacacggtgcatctgattggcggaacccaaacccttccgcagccctccc

SEQ ID NO: 10 - DNA, 122 bp, Homo sapiens
gtccctacttacttgctgttttagggcattttcagcgacttcactctcttctacctcaaacgccatccacttttggagacaggtaccactgcccctaggcagggacttgggagaggacctta

SEQ ID NO: 11 - DNA, 122 bp, Homo sapiens
tcccagaggccccccagcagcccccgagcagtccccggagcgctgggggactctgcagcgcgcaggtgaccacgcagagacagccccagcgcgatgaataattgaggcgcagagtggaatta

SEQ ID NO: 12 - DNA, 122 bp, Homo sapiens
ccgcccccagggtcccttcctatggggcagggcgggccctggccggcagcgattcaggaccgccccagagacggccaggagggcgtgggggaggggcgtggggcagaaacaggggtgtggag

SEQ ID NO: 13 - DNA, 122 bp, Homo sapiens
ctcgaccttgagaaccctgaacaattagtggcagggctggaatgagaatcctggtaaacccgcctcctaagccggtgttcggaattaccccgccagggcccgtgctgctcagtggtcctctc

SEQ ID NO: 14 - DNA, 122 bp, Homo sapiens
ttgctggtaataaaagcagaagtcacgtgctgaacgtgaaggtgagcagaggagaacttgcgatggcaaagttaaaaacaagaggagatgatggtcttggtgtggcacaggatgttaaaaaa

SEQ ID NO: 15 - DNA, 122 bp, Homo sapiens
ctagtccaaccgggtcattctacagatggagaaactgaggcccggagatgagcctcaacgcgtctaagtcacagaacaagtcagtggctatgtctctaaggctcaccgtggctgttggagtg

SEQ ID NO: 16 - DNA, 122 bp, Homo sapiens
ctccaccctcgggcattccgaaaagtcaaatcgagcttatgcactcaaatccaagtagggcggagccaaacctgtgtcctgggcgcctgccgggcaaggcgcggggactggacgggtcggaa

SEQ ID NO: 17 - DNA, 122 bp, Homo sapiens
actcgggttgggcccggcgacttgtgttttaaaagccctccgggtgattttgtgcatgctcgaccttgagaaccctgaacaattagtggcagggctggaatgagaatcctggtaaacccgcc

SEQ ID NO: 18 - DNA, 76 bp, Homo sapiens
ccgcggggaggggcgccggggcagcggccgggagctgcgccgcattcgcccgggacccctgctcctaggttcccta

SEQ ID NO: 19 - DNA, 77 bp, Homo sapiens
ccccgggacaagagcaccttctgtttccctgaagagacccggcctccctagagcagggccaccgccgccgcctgtgt

SEQ ID NO: 20 - DNA, 78 bp, Homo sapiens
cttggttctgcactgttgccaataaaaagctcttaaaaacgcattcgccaggctacagtgtttatttcctcctaacac

(19) United States
(12) Patent Application Publication
(10) Pub. No.: US 2016/0102363 A1
(43) Pub. Date: Apr. 14, 2016

(54) METHODS AND COMPOSITIONS FOR PREDICTING THERAPEUTIC EFFICACY OF KINASE INHIBITORS IN PATIENTS WITH MYELODYSPLASTIC SYNDROME OR RELATED DISORDERS

(71) Applicants: ONCONOVA THERAPEUTICS, INC., Newtown, PA (US); THE TRUSTEES OF COLUMBIA UNIVERSITY IN THE CITY OF NEW YORK, New York, NY (US)

(72) Inventors: BENJAMIN TYCKO, DEMAREST, NJ (US); AZRA RAZA, NEW YORK, NY (US); FRANCOIS WILHELM, PRINCETON, NJ (US)

(73) Assignees: ONCONOVA THERAPEUTICS, INC., NEWTOWN, NJ (US); THE TRUSTEES OF COLUMBIA UNVIERSITY IN THE CITY OF NEW YORK, NEW YORK, NY (US)

(21) Appl. No.: 14/893,935

(22) PCT Filed: May 28, 2014
(86) PCT No.: PCT/US14/39798

§ 371 Date: Nov. 24, 2015

Related U.S. Application Data
(60) Provisional application No. 61/829,754, filed on May 31, 2013.
(60) Provisional application No. 61/913,189, filed on Dec. 6, 2013.

(51) Int. Cl. C12Q 1/68; A61K 31/197

(52) U.S. Cl. CPC C12Q 1/6886; A61K 31/197; C12Q 2600/154; C12Q 2600/106

(57) ABSTRACT

The invention discloses a diagnostic method for predicting the therapeutic efficacy of a broad specificity kinase inhibitor in a subject with refractory cancer comprising determining the locus-specific DNA methylation profile of the subject, wherein the locus-specific DNA methylation profile predicts the therapeutic efficacy of a broad specificity kinase inhibitor for treatment of a subject with refractory cancer.

### CROSS REFERENCE TO RELATED APPLICATIONS

This application claims priority to U.S. Provisional Application No. 61/829,754, filed May 31, 2013; U.S. Provisional Application No. 61/913,189 filed Dec. 6, 2013; and PCT Patent Application No PCT/US2014/039798, filed May 28, 2014, the entire contents of each of which are incorporated herein by reference.

### FIELD OF THE INVENTION

The invention relates to methods for patient selection and predicting therapeutic efficacy of kinase inhibitors in patients with myelodysplastic syndrome. Specifically the diagnostic and prognostic methods are directed to use of a panel of DNA methylation biological markers to identify patients who are responsive to kinase inhibitors.

### I. BACKGROUND OF THE INVENTION

Cancer treatments, in general, have a higher rate of success if the cancer is diagnosed early and treatment is started earlier in the disease process. For the most part there is a direct relationship between improved prognosis and stage of disease at diagnosis for all forms of cancer. A significant number of tumors are classified as poorly or non-responsive to current therapeutic drugs or radiotherapy. Increasing the chemotherapeutic dosage or radiation dose not only fails to improve the therapeutic response, but also contributes to the development of side effects and resistance to therapy.

Bone marrow malignancies are clonal disorders resulting from neoplastic transformation of hematopoietic stem or progenitor cells Similar to their normal counterparts, transformed blood-forming cells remain dependent on signals from the hematopoiesis-regulating stromal environment for survival and proliferation. A review of the literature on stromal abnormalities in the leukemias, the myelodysplastic syndromes, and multiple myeloma reveals three principal mechanisms by which stromal derangements can contribute to the evolution of a neoplastic disease. In the simplest case, neoplastic blood-forming cells induce reversible changes in stroma function or composition which result in improved growth conditions for the malignant cells. In the second setting, functionally abnormal end cells derived from the malignant clone become an integral part of the stroma system, selectively stimulating the neoplastic cells and inhibiting normal blood cell formation. In the third condition, the emergence of a neoplastic cell population is the consequence of a primary stroma lesion characterized by inability to control regular blood cell formation (malignancy-inducing microenvironment).

The WHO classification system for hematopoietic tumors recognizes five categories of myeloid malignancies, including acute myeloid leukemia (AML), MDS (Myelodysplastic Syndrome), MPN (Myeloproliferative Neoplasm), MDS/MPN overlap, and PDGFR/FGFR1-rearranged myeloid/lymphoid neoplasms with eosinophilia. Myelodysplastic syndrome (MDS) and MPN are two groups of diseases in the family of bone marrow malignancies. MDS and MPN are not single diseases, but each encompasses a collection of hematopoietic and stem cell disorders.

The myelodysplastic syndromes (MDS, formerly known as preleukemia) are a diverse collection of hematological medical conditions that involve ineffective production (or dysplasia) of the myeloid class of blood cells. The WHO MDS category of diseases includes refractory anemia (RA), refractory anemia with ringed sideroblasts (RARS), refractory anemia with excess blasts (RAEB), refractory anemia with excess blasts in transformation (RAEB-T), and chronic myelomonocytic leukemia (CMML). Patients with MDS often develop severe anemia and require frequent blood transfusions. In most cases, the disease worsens and the patient develops cytopenias (low blood counts) caused by progressive bone marrow failure. In about one third of patients with MDS, the disease transforms into acute myelogenous leukemia (AML), usually within months to a few years.

Every year, at least 15,000 patients in the US are diagnosed with MDS (Goldberg et al, 2010; Rollison et al, 2006). The age at which most patients are diagnosed is between 60 and 75 years old. Survival of patients with MDS is dependent on the severity of their disease; on average, it is 3 to 5 years after initial diagnosis (Ma et al, 2007). Most patients succumb to complications of cytopenias (uncontrollable bleeding or infections). The disease may also progress to AML. Cases of AML that arise from prior MDS do not respond well to chemotherapy and have a poor prognosis.

Several hematologic conditions, including MDS, AML and MPNs have common features both in terms of the pathology and causal events. They also share common genetic determinants. For example, these maladies share anemia as a common feature. There have also been many cases of MDS/MPN overlap. MDS/MPN overlap disorders come in many variations: as a true overlap condition at initial presentation, with evidence of dysplasia of cellular elements and myeloproliferative components (such as fibrosis, hypercellularity, or organomegally); as MDS that takes on MPN features over time; or, conversely, as an MPN in which progressive marrow dysplasia develops. These disorders include chronic myelomonocytic leukemia (CMML), atypical (BCR-ABL1 negative) chronic myeloid leukemia, juvenile myelomonocytic leukemia, and MDS/MPNu1. Some MDS/MPN cases have JAK2 mutations (such as the provisional entity, refractory anemia with ring sideroblasts and thrombocytosis). The proliferative components of these disorders are related to abnormalities in the RAS/MAPK signaling pathways, and approximately 50 percent are associated with TET2 mutations.

While investigational drug therapies exist, there is currently not a curative drug treatment for most hematological cancers. Current treatment strategies for hematopoietic cancers include: allogeneic stem cell transplantation, Chemotherapy, Erythropoietin-stimulating agents (ESAs), blood transfusion, and DNA methyltransferase inhibitors.

Human cancer cells typically contain somatically altered genomes, characterized by mutation, amplification, or deletion of critical genes. In addition, the DNA template from human cancer cells often displays somatic changes in DNA methylation. See, e.g., E. R. Fearon, et al, Cell 61:759 (1990); P. A. Jones, et al, Cancer Res. 46:461 (1986); R. Holliday, Science 238:163 (1987); A. De Bustros, et al, Proc. Natl. Acad. Sci. USA 85:5693 (1988); P. A. Jones, et al, Adv. Cancer Res. 54:1 (1990); S. B. Baylin, et al, Cancer Cells 3:383 (1991); M. Makos, et al, Proc. Natl. Acad. Sci. USA 89:1929 (1992); N. Ohtani-Fujita, et al, Oncogene 8:1063 (1993).

DNA methylases transfer methyl groups from the universal methyl donor S-adenosyl methionine to specific sites on the DNA. Several biological functions have been attributed to the methylated bases in DNA. The most established biological function is the protection of the DNA from digestion by cognate restriction enzymes. This restriction modification phenomenon has, so far, been observed only in bacteria.

Mammalian cells, however, possess different methylases that exclusively methylate cytosine residues on the DNA that are 5′ neighbors of guanine (CpG). This methylation has been shown by several lines of evidence to play a role in gene activity, cell differentiation, tumorigenesis, X-chromosome inactivation, genomic imprinting and other major biological processes (Razin, A., H., and Riggs, R. D. eds. in DNA Methylation Biochemistry and Biological Significance, Springer-Verlag, N. Y., 1984).

In eukaryotic cells, methylation of cytosine residues that are immediately 5′ to a guanosine, occurs predominantly in CpG poor loci (Bird, A., Nature 321:209 (1986)). In contrast, discrete regions of CG dinucleotides called CpG islands (CGi) remain unmethylated in normal cells, except during X-chromosome inactivation and parental specific imprinting (Li, et al, Nature 366:362 (1993)) where methylation of 5′ regulatory regions can lead to transcriptional repression. For example, de novo methylation of the Rb gene has been demonstrated in a small fraction of retinoblastomas (Sakai, et al, Am. J. Hum. Genet., 48:880 (1991)), and a more detailed analysis of the VHL gene showed aberrant methylation in a subset of sporadic renal cell carcinomas (Herman, et al, Proc. Natl. Acad. Sci. U.S.A., 91:9700 (1994)). Expression of a tumor suppressor gene can also be abolished by de novo DNA methylation of a normally unmethylated 5′ CpG island. See, e.g., Issa, et al, Nature Genet. 7:536 (1994); Merlo, et al, Nature Med. 1:686 (1995); Herman, et al, Cancer Res., 56:722 (1996); Graff, et al, Cancer Res., 55:5195 (1995); Herman, et al, Cancer Res. 55:4525 (1995). A recent review outlines some of the challenges of implementing cancer sequencing in clinical oncology (Implementing personalized cancer genomics in clinical trials Richard Simon and Sameek Roychowdhury, Nature Reviews Drug Discovery Vol. 12, May 2013, 358-369, incorporated by reference in its entirety).

Several recent candidate gene and whole-exome approaches have yielded new insights into the potential genetic causes of MDS. These include EZH2 mutations (Ernst T et al: Nat Genet 42:722-726, 2010; Nikoloski G et al Nat Genet 42:665-667, 2010) and mutations in the spliceosome machinery (Yoshida K et al Nature 478:64-69, 2011; Papaemmanuil E et al N Engl J Med 365:1384-1395, 2011; Graubert T A et al Nat Genet 44:53-57, 2012). However, the challenge has been to delineate how this knowledge can be used to inform the care of patients with MDS. In a seminal article, Bejar et al (Bejar R et al N Engl J Med 364:2496-2506, 2011) performed extensive mutational profiling of a large cohort of patients with MDS and found that mutations in five genes, specifically ASXL1, EZH2, TP53, ETV6, and RUNX1, predicted for adverse outcome in MDS. More recently, they extended their genetic studies to patients with lower risk MDS and found that mutations in the same genes (with the exception of ETV6) were associated with independent, adverse, prognostic relevance in lower risk MDS (Bejar R et al J Clin Oncol 30:3376-3382, 2012). Consequently, there are now clinical tests for mutations in these specific genes available for clinicians and patients with MDS.

The recognition of epigenetic changes in DNA structure in MDS has shown that proper DNA methylation is critical in the regulation of proliferation genes, and the loss of DNA methylation control can lead to uncontrolled cell growth, and cytopenias. The recently approved DNA methyltransferase inhibitors take advantage of this mechanism by creating a more orderly DNA methylation profile in the hematopoietic stem cell nucleus, and thereby restore normal blood counts and retard the progression of MDS to acute leukemia.

The most important goals in treatment of hematopoietic cancers, in addition to prolonging survival, are development of higher hematologic responses and improvement in quality of life. Since hematopoietic cancers are biologically complex heterogeneous diseases, a single treatment strategy may not work for all patients. Accordingly, known therapies are not curative, and patients ultimately fail to respond over time. This failure of response leads to a poor prognosis where the average life expectancy is within few months. It would therefore be extremely beneficial if there were a way to predict whether a given patient with myelodysplastic syndrome would be likely to be therapeutically resistant or responsive to treatment with a kinase inhibitor.

Work from many investigators over the past two decades has clearly established that DNA methylation patterns are altered in human cancer cells, including in cases of myelodysplastic syndrome (MDS) and acute myeloid leukemia (AML). Such alterations have been utilized as biomarkers for cancer detection. However, there have been as yet no studies showing an ability of panels of differentially methylated genes to strongly predict patient-specific responses to anti-cancer or anti-MDS drugs. This situation is despite the fact that two classes of such drugs, DNA methylation inhibitors (e.g., Decitabine; 5aza-dC) and histone deacetylase inhibitors (e.g., Vorinostat) have anti-cancer effects and, in the case of Decitabine, beneficial effects in pre-malignant myelodysplastic syndrome, that are thought to be mediated through epigenetic mechanisms.

Accordingly, there is a long felt need for discovering new diagnostic methods for predicting in advance the therapeutic efficacy of kinase inhibitors in patients with myelodysplastic syndrome.

The present invention as disclosed and described herein provides diagnostic and therapeutic methods and compositions that can be used to predict the therapeutic efficacy of kinase inhibitors in patients with myelodysplastic syndrome.

### II. SUMMARY OF THE INVENTION

The present invention provides diagnostic methods and compositions for predicting therapeutic efficacy of a broad specificity kinase inhibitor in a subject with cancer.

In one aspect, the present invention provides compositions for determining the DNA methylation profile of a sample of a subject with cancer comprising a discrete panel of DNA methylation biological markers in a diagnostic method to distinguish between subjects who are resistant or responsive to a broad specificity kinase inhibitor.

In one embodiment, polynucleotide compositions are provided for determining the DNA methylation profile of a sample of a subject with refractory hematological cancer comprising a discrete panel of DNA methylation biological markers to distinguish between subjects who are resistant or responsive to a broad specificity kinase inhibitor, wherein the discrete panel of DNA methylation biological markers comprises the fifty differentially methylated gene biological markers listed in Tables 1, 2, 3, or 4 infra, or any sub-combination thereof, or fragments thereof comprising at least 16 contiguous bases.

In another aspect, the present invention also provides diagnostic methods comprising determining the DNA methylation profile of a sample of a subject with cancer and comparing the DNA methylation profile to a discrete panel of DNA methylation biological markers to distinguish between subjects who are resistant or responsive to a broad specificity kinase inhibitor.

In one embodiment, the invention provides a diagnostic method for predicting the therapeutic efficacy of broad specificity kinase inhibitors in a subject with refractory hematological cancer comprising determining the DNA methylation profile of a sample of a subject with refractory hematological cancer and comparing the DNA methylation profile to a discrete panel of DNA methylation biological markers to distinguish between subjects who are resistant or responsive to a broad specificity kinase inhibitor.

In yet another embodiment, the invention provides a diagnostic method for predicting the therapeutic efficacy of broad specificity kinase inhibitors in a subject with refractory cancer comprising: (a) obtaining an isolated test genomic DNA sample from a tissue; (b) subjecting the test genomic DNA sample to DNA methylation analysis whereby the DNA methylation profile of one or more CpG dinucleotide sequences is determined; and (c) comparing the DNA methylation profile of one or more CpG dinucleotide sequences of the test genomic DNA sample with that of corresponding sequences of a discrete panel of DNA methylation biological markers, wherein the therapeutic efficacy of a kinase inhibitor for treatment of a subject with refractory cancer is predicted in advance.

In a preferred embodiment, the invention provides a diagnostic method for predicting the therapeutic efficacy of broad specificity kinase inhibitors in a subject refractory hematological cancer comprising: (a) obtaining an isolated test genomic DNA sample from a tissue; (b) subjecting the test genomic DNA sample to DNA methylation analysis, whereby the DNA methylation profile of one or more CpG dinucleotide sequences is determined; and (c) comparing the DNA methylation profile of the one or more CpG dinucleotide sequences of the test sample with that of corresponding sequences of a discrete panel of DNA methylation biomarkers comprising the differentially methylated genes listed Tables 1, 2, 3, or 4 infra (or any sub-combination thereof), wherein the therapeutic efficacy of a broad specificity kinase inhibitor for treatment of a subject with refractory hematological cancer is predicted in advance.

In one embodiment of the diagnostic method, the broad specificity kinase inhibitor is a pharmaceutical composition comprising at least one compound of Formula 1

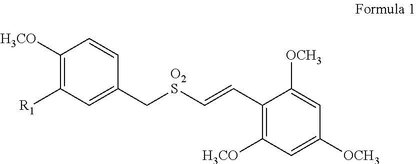

Where R₁ is selected from the group consisting of —NH₂, —NH—CH₂—COOH, —NH—CH(CH₃)—COOH, —NH—C(CH₃)₂—COOH, —NH—CH₂—CH₂—OH and —N—(CH₂CH₂OH)₂ a pharmaceutically acceptable salt of such a compound, an anticancer agent, or a combination thereof.

In a preferred embodiment of the diagnostic method, the hematopoietic cancer is myelodysplastic syndrome (MDS).

In a preferred embodiment of the diagnostic method, the hematopoietic cancer is refractory myelodysplastic syndrome (MDS).

In a preferred embodiment of the diagnostic method, the broad specificity kinase inhibitor is Rigosertib represented herein by Formula 1 A

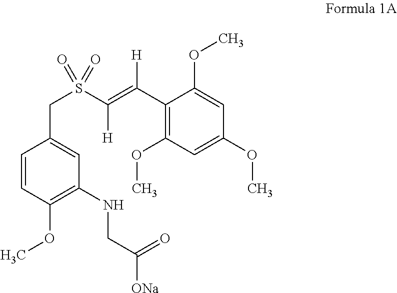

In one embodiment of the diagnostic method, the DNA methylation profile analysis utilizes an Illumina Infinium Human Methylation 450 BeadChip Array based upon a genome-wide analysis of methylation patterns to discover a discrete panel of predictive loci DNA methylation biological markers comprising the differentially methylated genes listed Tables 1, 2, 3, or 4 infra, or any sub-combination thereof.

In one embodiment, the DNA methylation profile analysis utilizes the Illumina Infinium Human Methylation 450 BeadChip to screen genomic DNA of bone marrow of patients with refractory MDS and the specific list of differentially methylated genes identified as being associated with refractory MDS is listed Tables 1, 2, 3, or 4, infra, or any sub-combination thereof).

In a preferred embodiment of the diagnostic method, the discrete panel of predictive loci DNA methylation biological markers comprising the differentially methylated genes listed Tables 1, 2, 3, or 4, infra, or any sub-combination thereof, is then validated with bisulphite DNA sequencing, which validated predictive loci DNA methylation biological markers can then be used in one or more clinical tests, wherein the therapeutic efficacy of a broad specificity kinase inhibitor for treatment of a subject with refractory MDS is predicted in advance.

In certain embodiments of the diagnostic method, the DNA methylation profiling is determined prior to, concomitant with, and/or subsequent to the administration of Rigosertib.

In one embodiment of the diagnostic method, the DNA methylation profile analysis comprises: (a) reacting the test genomic DNA sample with sodium bisulfite to convert unmethylated cytosine residues to uracil residues while leaving any 5-methylcytosine residues unchanged to create an exposed bisulfite-converted DNA sample having binding sites for primers specific for the bisulfite-converted DNA sample; (b) performing a PCR amplification procedure using top strand or bottom strand specific primers; (c) isolating the PCR amplification products; (d) performing a primer extension reaction using the gene specific primers for one or more of the differentially methylated genes listed in Tables 1, 2, 3, or 4, infra, dNTPs and Taq polymerase, wherein the primer comprises from about a 15-mer to about a 22-mer length primer sequence that is complementary to the bisulfite-converted DNA sample and terminates immediately 5′ of the cytosine residue of the one or more CpG dinucleotide sequences to be assayed; and (e) determining the locus specific DNA methylation profile of the one or more CpG dinucleotide sequences by determining the identity of the first primer-extended base against a panel of DNA methylation biomarkers, wherein the locus-specific DNA methylation profile predicts in advance the therapeutic efficacy of a broad specificity kinase inhibitor for treatment of a subject with refractory cancer.

In one embodiment of the diagnostic method, the dNTPs are labeled with a label selected from the group consisting of radiolabels and fluorescent labels, and wherein determining the identity of the first primer-extended base is by measuring incorporation of the labeled dNTPs.

In a further aspect, in addition to diagnosing the therapeutic efficacy of a broad specificity kinase inhibitor for treatment of a subject with refractory hematological cancer is predicted in advance and the diagnostic methods of the present invention can be further used to determine the prognosis or outcome of the cancer.

In a general aspect, the present invention provides for a method that comprises selection of the subject with refractory cancer, diagnosis of the refractory cancer, prognosis of the refractory cancer, treatment of the refractory cancer, or any combination thereof.

In yet another embodiment, the method of the invention provides for the selection of appropriate treatment regimens, including combination therapy protocols, for the selected and identified population of patients.

In yet another embodiment, the invention provides for combining kinase inhibitors with agents that interfere with methylation pathways to achieve optimal efficacy in patient subsets as identified by the present method.

In a further aspect, the invention provides a computer implemented diagnostic method for predicting in advance and distinguishing a subject's resistance or responsiveness to a broad spectrum kinase inhibitor for treatment of a subject with refractory cancer.

In a further aspect, the invention provides diagnostic method-based kits containing ingredients and assays for predicting in advance the resistance or responsiveness to a broad spectrum kinase inhibitor for treatment of subjects with refractory cancer.

### III. BRIEF DESCRIPTION OF THE DRAWINGS

FIG. 1 depicts the heat map obtained from the DNA methylation profiling of bone marrow samples from a clinically well characterized series of patients with refractory myelodysplastic syndrome using an Illumina Infinium Human Methylation 450K BeadChip Array.

FIG. 2 depicts the bisulphite DNA sequencing validation of the hyper-methylated state of the FOSB gene in Rigosertib non-responder patients with refractory MDS.

FIG. 3 depicts the bisulphite DNA sequencing validation of the hyper-methylated state of the CASZI gene in Rigosertib responder patients with refractory MDS.

### IV. DETAILED DESCRIPTION OF THE INVENTION

### Definitions

As used herein, “anticancer agents” are defined broadly to include agents that modulate the growth and/or metastasis of a cancer, treat or ameliorate one or more symptoms of a cancer, and/or treat or ameliorate one or more symptoms of secondary complications of the cancer.

As used herein, the terms “treat” and “treatment” are used interchangeably and are meant to indicate eradication of the disease, a postponement of development of a disorder and/or a reduction in the severity of symptoms that will or are expected to develop. The terms further include ameliorating existing symptoms, preventing additional symptoms, and ameliorating or preventing the underlying biological/medical causes of symptoms.

As used herein, “pharmaceutical composition” refers to a composition that contains at least one compound of Formula 1 or an agonist, antagonist, biologically active fragments, variants, analogs, isomers (structural isomers and stereoisomers and racemic mixtures) modified analogs, and functional analogs of at least one compound of Formula 1. The pharmaceutical composition of the invention may also contain additional anticancer agents as defined herein.

As used herein, “response” is defined by standard clinical criteria, importantly including amelioration of transfusion-dependent anemia, which is a major hallmark of myelodysplastic syndromes (MDS).

The inventors have discovered a discrete panel of DNA methylation biological markers which can be employed in a state-of-the-art personalized medicine approach. Specifically, the inventors have identified a discrete panel of sensitive and specific DNA methylation biological markers useful for predicting the therapeutic efficacy of kinase inhibitors in subjects with refractory MDS.

The present invention is thus based, in part, on the discovery that a discrete panel of DNA methylation biological markers can predict the therapeutic efficacy of kinase inhibitors in subjects with refractory hematological cancers. Specifically, the inventors have discovered that a specific panel of DNA methylation biological markers may be used on samples of subjects with refractory hematological cancers in a diagnostic method to predict in advance and distinguish between those subjects who are resistant or responsive to kinase inhibitors.

The discrete panel of DNA methylation biological markers for a particular cancer may be referred to collectively as the predictive DNA methylation signature for that cancer.

I. Diagnostic Methods

In one aspect of the present invention, diagnostic methods are provided for predicting the therapeutic efficacy of kinase inhibitors in a subject with refractory cancer comprising assaying a genomic DNA sample from a subject with refractory cancer and screening that DNA sample against a panel of locus-specific DNA methylation biological markers to determine locus specific DNA methylation profile patterns, wherein the locus-specific DNA methylation profile predicts the therapeutic efficacy of a broad specificity kinase inhibitor for treatment of a subject with refractory cancer.

In one embodiment, a diagnostic method is provided for predicting the therapeutic efficacy of a broad specificity kinase inhibitor in a subject with refractory cancer comprising: (a) obtaining a test genomic DNA sample from a test tissue of the subject; (b) analyzing the DNA methylation profile of the test genomic DNA sample, whereby the DNA methylation profile of one or more CpG dinucleotide sequences is determined; and (c) comparing the DNA methylation profile of the one or more CpG dinucleotide sequences of the test genomic DNA sample with corresponding sequences of a discrete panel of DNA methylation biomarkers to determine locus specific DNA methylation profile patterns, wherein the locus-specific DNA methylation profile of the test genomic DNA predicts the therapeutic efficacy of a broad specificity kinase inhibitor for treatment of a subject with refractory cancer.

In one preferred embodiment, the invention provides a diagnostic method for predicting the therapeutic efficacy of kinase inhibitors in a subject with refractory cancer comprising: (a) obtaining a test genomic DNA sample from a test tissue of the subject; (b) analyzing the DNA methylation profile of the test genomic DNA sample, whereby the DNA methylation profile of one or more CpG dinucleotide sequences is determined; and (c) comparing the DNA methylation profile of the one or more CpG dinucleotide sequences of the test genomic DNA sample with corresponding sequences of a discrete panel of DNA methylation biomarkers comprising the differentially methylated genes listed Tables 1, 2, 3, or 4, infra, or any sub-combination thereof, to determine locus specific DNA methylation patterns, wherein the locus-specific DNA methylation profile of the test genomic DNA predicts the therapeutic efficacy of a broad specificity kinase inhibitor for treatment of a subject with refractory cancer.

In one embodiment, the DNA methylation comprises methylation of cytosine residues that are immediately 5′ to a guanosine (i.e., 5-meC). In another embodiment, the DNA methylation comprises a modified methylation of cytosine residues that are immediately 5′ to a guanosine (i.e., 5-HyroxyMeC, 5-formylMeC and 5-carboxyMeC, 3-Methylcytosine (3-mC), or any combination thereof).

In one embodiment the broad specificity kinase inhibitor comprises PT 3-kinases, polo-like kinase 1 (PLK-1), or both.

In one embodiment of the diagnostic method of the present invention, the kinase inhibitor is a pharmaceutical composition comprising at least one compound of Formula 1

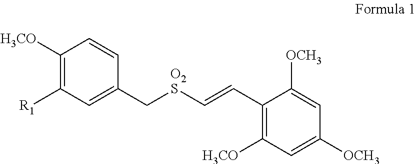

Where R₁ is selected from the group consisting of —NH₂, —NH—CH₂—COOH, —NH—CH(CH₃)—COOH, —NH—C(CH₃)₂—COOH, —NH—CH₂—CH₂—OH and —N—(CH₂CH₂OH)₂ a pharmaceutically acceptable salt of such a compound, an anticancer agent, or a combination thereof.

In a preferred embodiment of the diagnostic method, the kinase inhibitor is Rigosertib. Rigosertib is also known as ON 01910.Na and/or Estybon.

In one embodiment of the diagnostic method, the test tissue is a cancer tissue or a putative cancer tissue derived from a subject, and the reference DNA methylation biomarker profile is derived from MDS bone marrow biopsy samples, wherein the locus-specific DNA methylation profile predicts the therapeutic efficacy of a broad specificity kinase inhibitor for treatment of a subject with refractory cancer.

In one embodiment of the diagnostic method, the refractory cancer comprises hematopoietic cancer, pancreatic cancer, head and neck cancer, cutaneous tumors, acute lymphoblastic leukemia (ALL), minimal residual disease (MRD) in acute lymphoblastic leukemia (ALL), acute myeloid leukemia (AML), lung cancer, breast cancer, ovarian cancer, prostate cancer, colon cancer, melanoma or other hematological diseases and solid tumors.

In a preferred embodiment of the diagnostic method, the hematopoietic refractory cancer is myelodysplastic syndrome.

In yet another preferred embodiment of the diagnostic method, the hematopoietic refractory cancer comprises acute myeloid leukemia (AML), MPN (Myeloproliferative Neoplasm), MDS/MPN overlap, and PDGFR/FGFR1-rearranged myeloid/lymphoid neoplasms with eosinophilia, related disorders, or any combination thereof.

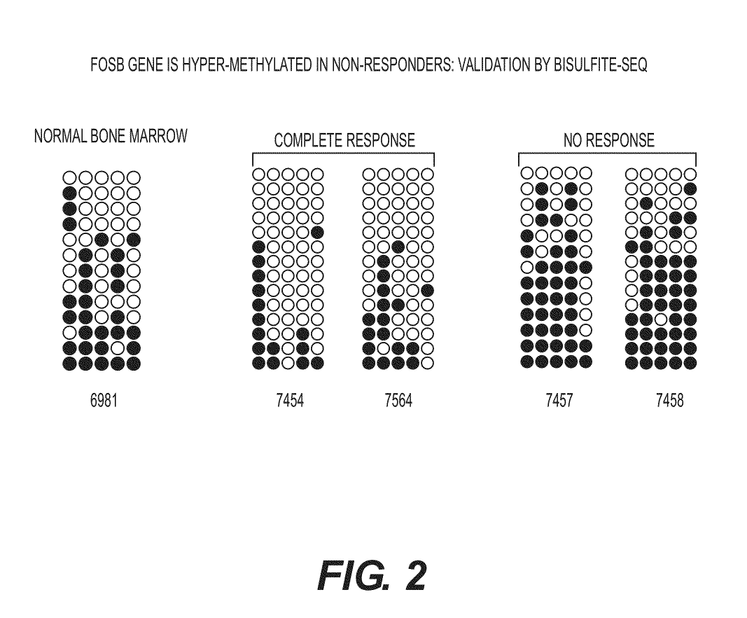

In another embodiment of the diagnostic method, the cancer comprises myelodysplastic syndrome, and the at least one DNA methylation profile FOSB gene marker is hyper-methylated in Rigosertib non-responder patients with refractory MDS, wherein the hyper-methylated status of the DNA methylation profile FOSB gene marker is validated though bisulphite DNA sequencing (c.f., FIG. 2).

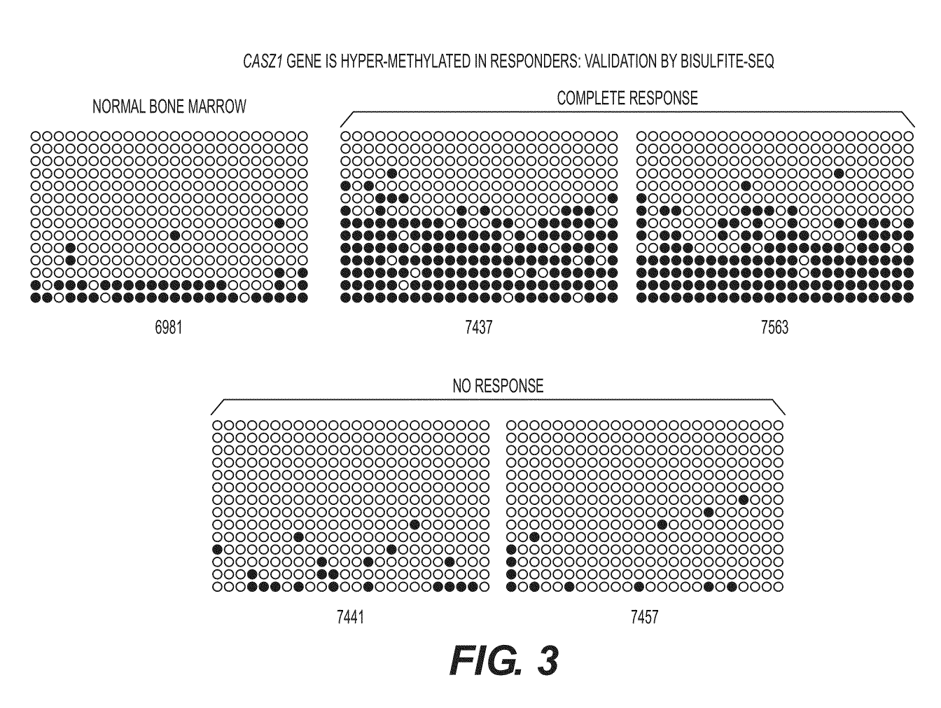

In yet another embodiment of the diagnostic method, the cancer comprises myelodysplastic syndrome, and the at least one DNA methylation profile CASZI gene marker is hyper-methylated in Rigosertib responder patients with refractory MDS, wherein the hyper-methylated status of the DNA methylation profile CASZI gene marker is validated though bisulphite DNA sequencing (c.f., FIG. 3).

In a further aspect, the invention provides a diagnostic method-based kit containing assays and ingredients for determining the DNA methylation profile of a test genomic DNA sample and comparing the DNA methylation profile of the one or more CpG dinucleotide sequences of the test genomic DNA sample with the corresponding sequences of a discrete panel of DNA methylation biomarkers comprising one or more of the differentially methylated genes listed Tables 1, 2, 3, or 4, infra, or any sub-combination thereof, wherein the locus-specific DNA methylation profile predicts the therapeutic efficacy of a broad specificity kinase inhibitor for treatment of a subject with refractory cancer.

In yet another embodiment, the invention provides a diagnostic method for predicting the therapeutic efficacy of broad specificity kinase inhibitors in a subject with refractory hematological cancer comprising the use of DNA methylation profiles of genes, the mutations, or altered expressions, of which are associated with an increased prevalence of certain hematological cancers or hematopoietic disorders as a discrete panel of DNA methylation biological markers to screen for and predict in advance the therapeutic efficacy of a broad specificity kinase inhibitor for treatment of a subject with refractory cancers.

In one embodiment, the kit comprises a discrete panel of DNA methylation biomarkers comprising one or more of the differentially methylated genes listed Tables 1, 2, 3, or 4, infra, or any sub-combination thereof, one or more pairs of polynucleotide primers capable of specifically amplifying at least a portion of a DNA region of a test genomic DNA sample, wherein the primers are designed based upon one or more of the differentially methylated genes listed in Tables 1, 2, 3, or 4, infra, and instructions for use.

In a further aspect, the invention provides computer-implemented diagnostic methods for determining the DNA methylation profile of a test genomic DNA sample and comparing the DNA methylation profile of the one or more CpG dinucleotide sequences of the test genomic DNA sample with the corresponding sequences of a discrete panel of DNA methylation biomarkers comprising one or more of the differentially methylated genes listed Tables 1, 2, 3, or 4, infra, or any sub-combination thereof, wherein the locus-specific DNA methylation profile predicts the therapeutic efficacy of a broad specificity kinase inhibitor for treatment of a subject with refractory cancer.

In another aspect, the invention provides computer program products comprising a computer readable medium encoded with program code for receiving a methylation value representing the DNA methylation profile of a test genomic DNA sample; and program code for comparing the DNA methylation profile of the one or more CpG dinucleotide sequences of the test genomic DNA sample with the corresponding sequences of a discrete panel of DNA methylation biomarkers comprising one or more of the differentially methylated genes listed in Tables 1, 2, 3, or 4, infra, or any sub-combination thereof, wherein the locus-specific DNA methylation profile predicts the therapeutic efficacy of a broad specificity kinase inhibitor for treatment of a subject with refractory cancer.

II. DNA Methylation Biomarkers

The DNA region to be assayed to determine the DNA methylation profile state is obtained from samples from subjects with refractory cancers.

In one embodiment of the diagnostic method, the biological sample can be from any body fluid or tissue of the subject with refractory cancer.

In some embodiments of the diagnostic method, the biological sample is obtained from blood serum, blood plasma, fine needle aspirate of the breast, biopsy of the breast, ductal fluid, ductal lavage, feces, urine, sputum, saliva, semen, lavages, or tissue biopsy, such as biopsy of the lung, bronchial lavage or bronchial brushings in the case of lung cancer. In some embodiments, the sample is from a tumor or polyp.

In yet other embodiments of the diagnostic method, the biological sample is obtained from peripheral blood samples (i.e., after CD34 separation).

In some embodiments, the sample is a biopsy from lung, kidney, liver, ovarian, head, neck, thyroid, bladder, cervical, colon, endometrial, esophageal, prostate or skin tissue. In some embodiments, the sample is from skin punches, cell scrapes, washings, or resected tissues. In yet other embodiments, the biological sample is selected from whole blood, buffy coat, isolated mononuclear cells, plasma, serum, or bone marrow.

In one embodiment of the diagnostic method, the DNA region to be assayed to determine the DNA methylation profile state is obtained from in samples from subjects with refractory cancers comprises a nucleic acid including one or more methylation sites of interest (e.g., a cytosine, a “microarray feature,” or an amplicon amplified from select primers) and flanking nucleic acid sequences (i.e., “wingspan”) of up to 4 kilobases (kb) in either or both of the 3′ or 5′ direction from the amplicon. This range corresponds to the lengths of DNA fragments obtained by randomly fragmenting the DNA before screening for differential methylation between DNA in two or more samples.

In some embodiments of the diagnostic methods, the wingspan of the one or more DNA regions is about 0.5 kb, 0.75 kb, 1.0 kb, 1.5 kb, 2.0 kb, 2.5 kb, 3.0 kb, 3.5 kb or 4.0 kb in both 3′ and 5′ directions relative to the sequence represented by the microarray feature. The methylation sites in a DNA region can reside in non-coding transcriptional control sequences (e.g., promoters, enhancers, etc.) or in coding sequences, including introns and exons of the differentially methylated genes listed in Tables 1, 2, 3, or 4, infra.

In some embodiments of the diagnostic methods, the methods comprise detecting the methylation status in the promoter regions (e.g., comprising the nucleic acid sequence that is about 1.0 kb, 1.5 kb, 2.0 kb, 2.5 kb, 3.0 kb, 3.5 kb or 4.0 kb 5′ from the transcriptional start site through to the transcriptional start site) of one or more of the DNA methylation biomarker genes listed in Tables 1, 2, 3, or 4, infra. The DNA regions of the DNA methylation biomarker genes listed in Tables 1, 2, 3, or 4, infra also include naturally occurring variants, including for example, variants occurring in different subject populations and variants arising from single nucleotide polymorphisms (SNPs). SNPs encompass insertions and deletions of varying size and simple sequence repeats, such as dinucleotides and tri-nucleotide repeats. Variants include nucleic acid sequences from the same DNA region (e.g., as set forth in Tables 1, 2, 3, or 4, infra or that can be identified from the chromosome and physical position as for each DNA methylation biomarker gene) sharing at least 90%, 95%, 98%, 99% sequence identity, i.e., having one or more deletions, additions, substitutions, inverted sequences, etc., relative to the DNA regions described herein.

In some embodiments of the diagnostic methods, the DNA methylation state of more than one DNA region (or a portion thereof) in samples from subjects with refractory cancers is detected. In some embodiments, the DNA methylation status of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, or 97 of the DNA regions in samples from subjects with refractory cancers is determined.

In some embodiments of the diagnostic methods, the presence or absence or quantity of DNA methylation of the chromosomal DNA within a DNA region or portion thereof (e.g., at least one cytosine) selected from is detected in samples from subjects with refractory cancers and compared with a panel of DNA methylation biological markers to predict the therapeutic efficacy of a broad specificity kinase inhibitor for treatment of a subject with refractory cancers. Portions of the differentially methylated DNA regions described herein will comprise at least one potential DNA methylation site (i.e., a cytosine) and can in some embodiments generally comprise 2, 3, 4, 5, 10, or more potential methylation sites.

In some embodiments of the diagnostic methods, the methylation status of all cytosines within at least 20, 50, 100, 200, 500 or more contiguous base pairs of the differentially methylated DNA region are determined.

In one embodiment of the discrete panel of DNA methylation biological markers, the panel of DNA methylation biological markers used to predict the therapeutic efficacy of a broad specificity kinase inhibitor for treatment of a subject with refractory cancers comprises one or more of the DNA methylation biomarker genes listed in Tables 1, 2, 3, or 4, infra.

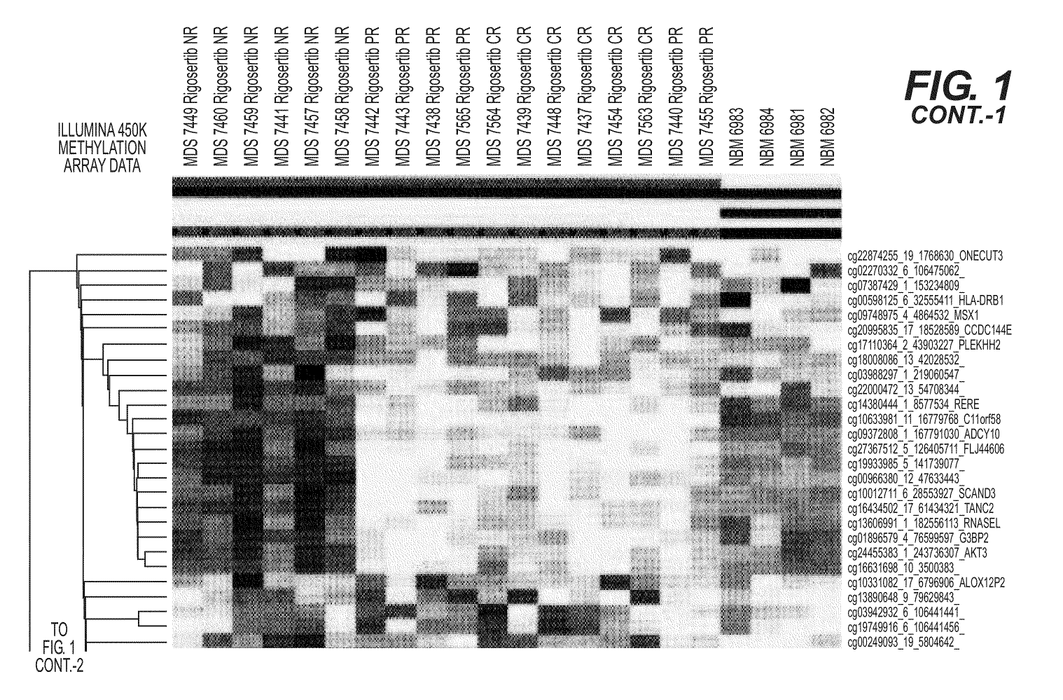

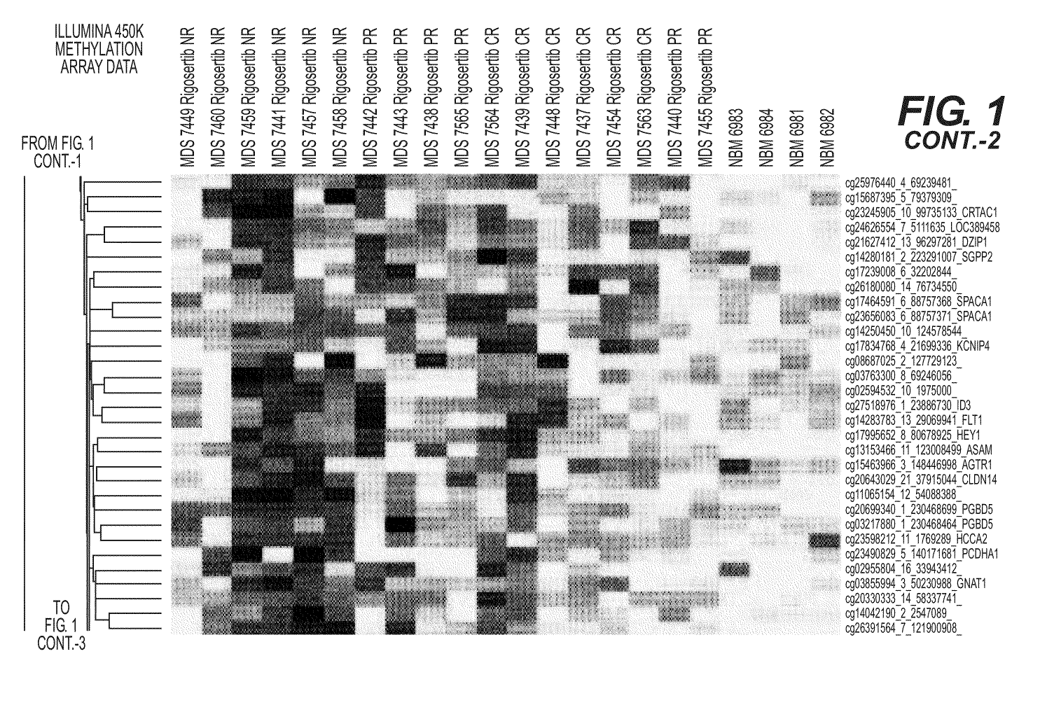

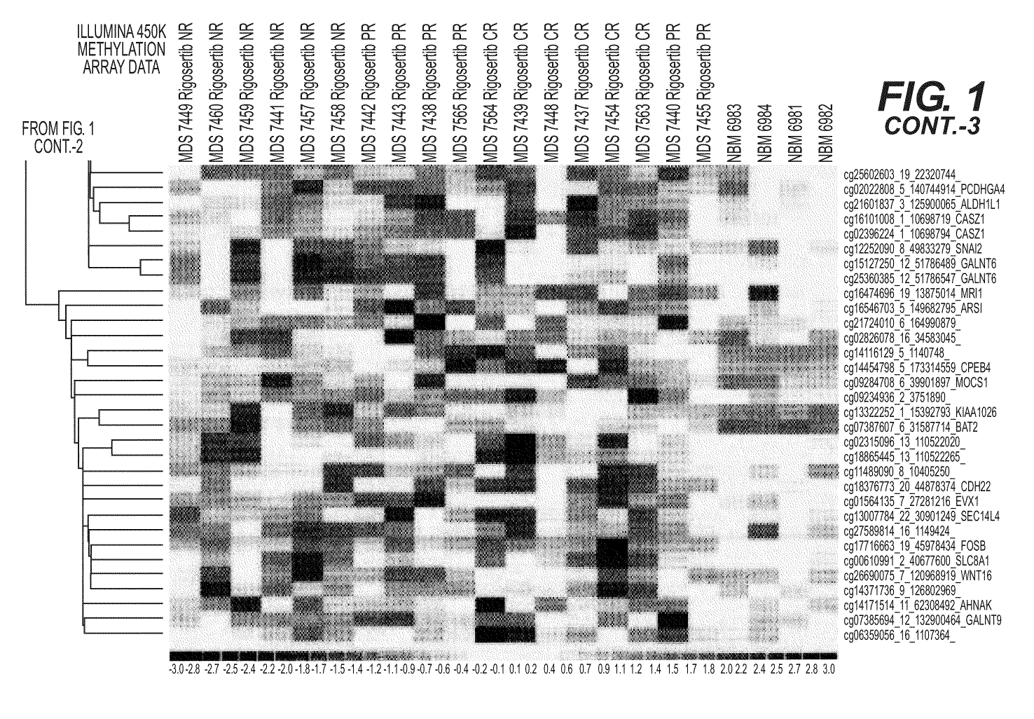

In a preferred embodiment of the diagnostic method of the present invention, the panel of DNA methylation biological markers used to predict the therapeutic efficacy of a broad specificity kinase inhibitor for treatment of a subject with a refractory cancer selected from the group consisting of acute myeloid leukemia (AML), MDS (Myelodysplastic Syndrome) MPN (Myeloproliferative Neoplasm), MDS/MPN overlap, and PDGFR/FGFR1-rearranged myeloid/lymphoid neoplasms with eosinophilia, or related disorder comprises those DNA methylation biological markers associated with the differentially methylated genes RERE, CASZ1, KIAA1026, ID3, ADCY10, RNASEL, PGBD5, AKT3, SLC8Al, PLEKHH2, SGPP2, GNAT1, ALDH1L1, AGTR1, MSX1, KCNIP4, G3BP2, FLJ44606, PCDHA1, PCDHGA4, ARSI, CPEB4, SCAND3, BAT2, HLA-DRB1, MOCS1, SPACA1, LOC389458, EVX1, WNT16, SNAI2, HEY1, CRTAC1, HCCA2, C11orf58, AHNAK, ASAM, GALNT6, GALNT9, FLT1, DZIP1, ALOX12P2, CCDC144B, TANC2, ONECUT3, MRI1, FOSB, CDH22, CLDN14, and SEC14L4, any variants thereof, or any combination thereof (cf. FIG. 1 and Tables 1, 2, 3, or 4, infra).

In yet another preferred embodiment of the diagnostic method of the present invention, the panel of DNA methylation biological markers used to predict the therapeutic efficacy of a broad specificity kinase inhibitor for treatment of a subject with refractory cancers comprises the DNA methylation biological markers specifically associated with the CASZ1 and FOSB genes (cf. FIG. 1 and Tables 1, 2, 3, or 4, infra) as validated by bisulphite DNA sequencing.

III Methods to Determine DNA Methylation Profiles

A variety of genome scanning methods may be used to determine the DNA methylation profile in cancer cells. For example, one method involves restriction landmark genomic scanning (Kawai et al, Mol. Cell. Biol. 14:7421-7427, 1994), and another example involves methylation-sensitive arbitrarily primed PCR (Gonzalgo et al, Cancer Res. 57:594-599, 1997). Changes in methylation patterns at specific CpG sites have been monitored by digestion of genomic DNA with methylation-sensitive restriction enzymes followed by Southern analysis of the regions of interest (digestion-Southern method). Genomic sequencing has been simplified for analysis of DNA methylation patterns and 5-methylcytosine distribution by using bisulfite treatment (Frommer et al, Proc. Natl. Acad. Sci. USA 89: 1827-1831, 1992). In addition, other techniques have been reported which utilize bisulfite treatment of DNA as a starting point for methylation analysis. These include methylation-specific PCR (MSP) (Herman et al Proc. Natl. Acad Sci. USA 93:9821-9826, 1992); and restriction enzyme digestion of PCR products amplified from bisulfite-converted DNA (Sadri and Hornsby, Nucl. Acids Res. 24: 5058-5059, 1996; and Xiong and Laird, Nucl. Acids Res. 25: 2532-2534, 1997).

PCR techniques may be used for detection of gene mutations (Kuppuswamy et al, Proc. Natl. Acad. Sci. USA 88:1143-1147, 1991) and quantitation of allelic-specific expression (Szabo and Mann, Genes Dev. 9:3097-3108, 1995; and Singer-Sam et al, PCR Methods Appl. 1:160-163, 1992). Such techniques use internal primers, which anneal to a PCR-generated template and terminate immediately 5′ of the single nucleotide to be assayed.

DNA methylation microarrays such the 1,505 CpG (Illumina GoldenGate DNA Methylation BeadArray)((Bibikova M et al Genome Res 2006; 16:383-93; Christensen B C et al PLoS Genet 2009; 5:1000602; Byun H M et al Hum Mol Genet 2009; 18:4808-17; Martinez R et al Epigenetics 2009; 4:255-64), and 27,000 CpG (Illumina Infinium HumanMethylation27 BeadChip)(Kanduri M et al Blood 2010; 115:296-305; Bork S et al Aging Cell 2010; 9:54-63; Teschendorff A E et al Genome Res 2010; 20:440-6; Rakyan V K et al Genome Res 2010; 20:434-9) and the launch of a new 450,000 CpG site platform for DNA methylation studies (Illumina Infinium HumanMethylation450 BeadChip) microarrays may be used to address the DNA methylation status of DNA regions. The 450K DNA methylation microarray has recently been validated from a biological, functional and technical standpoint using colorectal cancer and DNA methylation models (Sandoval et al Epigenetics 6:6, 692-702; June 2011).

Accordingly, in one embodiment of the diagnostic method, the validation of the DNA methylation profile of the diagnostic method of the present invention comprises a validation method selected from the group consisting of DNA sequencing using bisulfite treatment, restriction landmark genomic scanning, methylation-sensitive arbitrarily primed PCR, Southern analysis using a methylation-sensitive restriction enzyme, methylation-specific PCR, restriction enzyme digestion of PCR products amplified from bisulfite-converted DNA, and combinations thereof.

### V. EXAMPLES

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### Example 1

### Predictive Loci DNA Methylation Signature Profile (Illumina 450K Beadchips) of MDS Bone Marrow Biopsy Samples

Methods:

This study sought to identify DNA methylation markers that can predict the therapeutic response to a different, and an even more widely pertinent, class of anti-cancer drugs, namely broad-specificity tyrosine kinase inhibitors (TKI's) or a broad specificity kinase inhibitors. As used herein, “response” is defined by standard clinical criteria, importantly including amelioration of transfusion-dependent anemia, which is a major hallmark of myelodysplastic syndromes (MDS).

Patients with refractory MDS were biopsied and their bone marrow precursor biopsy samples subjected to DNA methylation profile analysis using a discrete panel of DNA methylation biomarkers comprising one or more genes selected from the group consisting of the differentially methylated genes listed in FIG. 1 and Tables 1, 2, 3, or 4, infra. The hyper-methylated status of the DNA methylation profile marker of the FOSB and CASZI genes was then independently validated though bisulphite DNA sequencing.

TABLE 1
Predictive Loci DNA methylation signature profile (Illumina 450K Beadchips) of MDS
bone marrow biopsy samples to determine whether locus-specific DNA methylation patterns
can predict response to a broad specificity kinase inhibitor drug (Rigosertib)

| Illumina Ref. | | Base Pair | | Baseline | Experiment | Fold | Difference | t | |
|---|---|---|---|---|---|---|---|---|---|
| Citation | Chromosome | Position | Gene Name | Mean | Mean | Change | of Means | Statistic | P Value |
| cg14380444 | 1 | 8577534 | RERE | 0.52 | 0.34 | −1.53 | −0.18 | −3.901 | 0.006607 |
| cg16101008 | 1 | 10698719 | CASZ1 | 0.58 | 0.39 | −1.51 | −0.2 | −3.7 | 0.004281 |
| cg02396224 | 1 | 10698794 | CASZ1 | 0.44 | 0.24 | −1.86 | −0.2 | −3.457 | 0.00932 |
| cg13322252 | 1 | 15392793 | KIAA1026 | 0.24 | 0.43 | 1.82 | 0.2 | 3.428 | 0.009337 |
| cg27518976 | 1 | 23886730 | ID3 | 0.58 | 0.35 | −1.66 | −0.23 | −4.017 | 0.003622 |
| cg07387429 | 1 | 153234809 | | 0.67 | 0.46 | −1.44 | −0.21 | −4.163 | 0.003438 |
| cg09372808 | 1 | 167791030 | ADCY10 | 0.58 | 0.38 | −1.5 | −0.19 | −4.963 | 0.001145 |
| cg13606991 | 1 | 182556113 | RNASEL | 0.64 | 0.48 | −1.34 | −0.16 | −4.4 | 0.001597 |
| cg03988297 | 1 | 219060547 | | 0.48 | 0.31 | −1.59 | −0.18 | −3.454 | 0.006744 |
| cg03217880 | 1 | 230468464 | PGBD5 | 0.64 | 0.48 | −1.34 | −0.16 | −4.751 | 0.000966 |
| cg20699340 | 1 | 230468699 | PGBD5 | 0.65 | 0.46 | −1.41 | −0.19 | −5.068 | 0.003163 |
| cg24455383 | 1 | 243736307 | AKT3 | 0.65 | 0.47 | −1.38 | −0.18 | −5.226 | 0.001514 |
| cg14042190 | 2 | 2547089 | | 0.8 | 0.48 | −1.67 | −0.32 | −3.752 | 0.005105 |
| cg09234936 | 2 | 3751890 | | 0.46 | 0.7 | 1.54 | 0.25 | 3.651 | 0.004462 |
| cg00610991 | 2 | 40677600 | SLC8A1 | 0.28 | 0.63 | 2.28 | 0.36 | 4.651 | 0.001061 |
| cg17110364 | 2 | 43903227 | PLEKHH2 | 0.59 | 0.37 | −1.6 | −0.22 | −4.717 | 0.000857 |
| cg08687025 | 2 | 127729123 | | 0.46 | 0.3 | −1.5 | −0.15 | −3.602 | 0.004838 |
| cg14280181 | 2 | 223291007 | SGPP2 | 0.56 | 0.35 | −1.62 | −0.21 | −3.522 | 0.008096 |
| cg03855994 | 3 | 50230988 | GNAT1 | 0.59 | 0.35 | −1.69 | −0.24 | −7.003 | 0.000049 |
| cg21601837 | 3 | 125900065 | ALDH1L1 | 0.46 | 0.29 | −1.59 | −0.17 | −3.275 | 0.009253 |
| cg15463966 | 3 | 148446998 | AGTR1 | 0.41 | 0.24 | −1.7 | −0.17 | −5.666 | 0.00025 |
| cg09748975 | 4 | 4864532 | MSX1 | 0.47 | 0.31 | −1.55 | −0.17 | −3.419 | 0.006575 |
| cg17834768 | 4 | 21699336 | KCNIP4 | 0.34 | 0.16 | −2.15 | −0.18 | −3.908 | 0.007182 |
| cg25976440 | 4 | 69239481 | | 0.41 | 0.21 | −1.92 | −0.2 | −3.959 | 0.004791 |
| cg01896579 | 4 | 76599597 | G3BP2 | 0.62 | 0.46 | −1.34 | −0.16 | −5.349 | 0.000672 |
| cg14116129 | 5 | 1140748 | | 0.28 | 0.44 | 1.56 | 0.16 | 3.553 | 0.00593 |
| cg15687395 | 5 | 79379309 | | 0.7 | 0.43 | −1.63 | −0.27 | −3.705 | 0.007239 |
| cg27367512 | 5 | 126405711 | FLJ44606 | 0.8 | 0.63 | −1.27 | −0.17 | −8.384 | 0.00001 |
| cg23490829 | 5 | 140171681 | PCDHA1 | 0.66 | 0.4 | −1.64 | −0.26 | −3.83 | 0.0034 |
| cg02022808 | 5 | 140744914 | PCDHGA4 | 0.4 | 0.23 | −1.71 | −0.17 | −3.585 | 0.006888 |
| cg19933985 | 5 | 141739077 | | 0.6 | 0.38 | −1.56 | −0.21 | −8.318 | 0.00017 |
| cg16546703 | 5 | 149682795 | ARSI | 0.19 | 0.34 | 1.78 | 0.15 | 3.925 | 0.003799 |
| cg14454798 | 5 | 173314559 | CPEB4 | 0.48 | 0.72 | 1.5 | 0.24 | 5.146 | 0.001528 |
| cg10012711 | 6 | 28553927 | SCAND3 | 0.74 | 0.57 | −1.31 | −0.18 | −5.326 | 0.000485 |
| cg07387607 | 6 | 31587714 | BAT2 | 0.18 | 0.35 | 1.95 | 0.17 | 3.66 | 0.004386 |
| cg17239008 | 6 | 32202844 | | 0.51 | 0.3 | −1.68 | −0.21 | −4.561 | 0.00253 |
| cg00598125 | 6 | 32555411 | HLA-DRB1 | 0.36 | 0.12 | −3.06 | −0.24 | −5.51 | 0.000272 |
| cg09284708 | 6 | 39901897 | MOCS1 | 0.35 | 0.52 | 1.5 | 0.17 | 3.322 | 0.009105 |
| cg17464591 | 6 | 88757368 | SPACA1 | 0.76 | 0.54 | −1.41 | −0.22 | −4.241 | 0.002697 |
| cg23656083 | 6 | 88757371 | SPACA1 | 0.84 | 0.61 | −1.37 | −0.23 | −4.833 | 0.001051 |
| cg03942932 | 6 | 106441441 | | 0.57 | 0.31 | −1.82 | −0.26 | −3.707 | 0.00451 |
| cg19749916 | 6 | 106441456 | | 0.6 | 0.35 | −1.73 | −0.25 | −3.554 | 0.006023 |
| cg02270332 | 6 | 106475062 | | 0.57 | 0.38 | −1.5 | −0.19 | −3.624 | 0.004684 |
| cg21724010 | 6 | 164990879 | | 0.44 | 0.59 | 1.36 | 0.15 | 4.636 | 0.001474 |
| cg24626554 | 7 | 5111635 | LOC389458 | 0.43 | 0.2 | −2.15 | −0.23 | −3.379 | 0.00937 |
| cg01564135 | 7 | 27281216 | EVX1 | 0.3 | 0.53 | 1.81 | 0.24 | 6.729 | 0.000607 |
| cg26690075 | 7 | 120968919 | WNT16 | 0.27 | 0.48 | 1.74 | 0.2 | 3.689 | 0.004744 |
| cg26391564 | 7 | 121900908 | | 0.43 | 0.18 | −2.39 | −0.25 | −4.362 | 0.001469 |
| cg11489090 | 8 | 10405250 | | 0.3 | 0.54 | 1.82 | 0.25 | 5.082 | 0.000786 |
| cg12252090 | 8 | 49833279 | SNAI2 | 0.58 | 0.4 | −1.45 | −0.18 | −3.612 | 0.004752 |
| cg03763300 | 8 | 69246056 | | 0.73 | 0.5 | −1.45 | −0.22 | −5.262 | 0.000565 |
| cg17995652 | 8 | 80678925 | HEY1 | 0.51 | 0.33 | −1.52 | −0.18 | −3.461 | 0.007731 |
| cg13890649 | 9 | 79629843 | | 0.43 | 0.27 | −1.58 | −0.16 | −3.264 | 0.008751 |
| cg14371736 | 9 | 126802969 | | 0.28 | 0.49 | 1.74 | 0.21 | 4.104 | 0.002507 |
| cg02594532 | 10 | 1975000 | | 0.38 | 0.22 | −1.75 | −0.16 | −4.525 | 0.001482 |
| cg16631698 | 10 | 3500383 | | 0.54 | 0.37 | −1.45 | −0.17 | −4.047 | 0.005888 |
| cg23245905 | 10 | 99735133 | CRTAC1 | 0.59 | 0.39 | −1.54 | −0.21 | −3.233 | 0.008971 |
| cg14250450 | 10 | 124578544 | | 0.71 | 0.51 | −1.39 | −0.2 | −4.249 | 0.002413 |
| cg23598212 | 11 | 1769289 | HCCA2 | 0.78 | 0.58 | −1.34 | −0.2 | −6.041 | 0.000507 |
| cg10633981 | 11 | 16779768 | C11orf58 | 0.6 | 0.44 | −1.37 | −0.16 | −10.836 | 0.000003 |
| cg14171514 | 11 | 62308492 | AHNAK | 0.3 | 0.53 | 1.76 | 0.23 | 3.414 | 0.008433 |
| cg13153466 | 11 | 123008499 | ASAM | 0.64 | 0.35 | −1.85 | −0.29 | −4.812 | 0.001039 |
| cg00966380 | 12 | 47633443 | | 0.8 | 0.64 | −1.25 | −0.16 | −9.615 | 0.000009 |
| cg15127250 | 12 | 51786489 | GALNT6 | 0.48 | 0.26 | −1.88 | −0.23 | −3.329 | 0.007752 |
| cg25360385 | 12 | 51786547 | GALNT6 | 0.66 | 0.37 | −1.78 | −0.29 | −4.104 | 0.002322 |
| cg11065154 | 12 | 54088388 | | 0.65 | 0.41 | −1.58 | −0.24 | −3.525 | 0.008389 |
| cg07385694 | 12 | 132900464 | GALNT9 | 0.67 | 0.91 | 1.35 | 0.24 | 4.809 | 0.000767 |
| cg14283783 | 13 | 29069941 | FLT1 | 0.41 | 0.25 | −1.61 | −0.15 | −4.511 | 0.001312 |
| cg18008086 | 13 | 42028532 | | 0.63 | 0.45 | −1.39 | −0.18 | −3.96 | 0.006651 |
| cg22000472 | 13 | 54708344 | | 0.65 | 0.47 | −1.39 | −0.19 | −6.193 | 0.000342 |
| cg21627412 | 13 | 96297281 | DZIP1 | 0.34 | 0.18 | −1.94 | −0.17 | −3.477 | 0.007703 |
| cg02315096 | 13 | 110522020 | | 0.31 | 0.54 | 1.72 | 0.22 | 5.212 | 0.000409 |
| cg18865445 | 13 | 110522265 | | 0.29 | 0.58 | 2.02 | 0.29 | 4.598 | 0.001007 |
| cg20330333 | 14 | 58337741 | | 0.37 | 0.14 | −2.6 | −0.22 | −4.76 | 0.003559 |
| cg26180080 | 14 | 76734550 | | 0.4 | 0.22 | −1.83 | −0.18 | −3.174 | 0.009944 |
| cg06359056 | 16 | 1107364 | | 0.36 | 0.58 | 1.59 | 0.21 | 3.677 | 0.005909 |
| cg27589814 | 16 | 1149424 | | 0.29 | 0.44 | 1.52 | 0.15 | 4.419 | 0.001767 |
| cg02955804 | 16 | 33943412 | | 0.63 | 0.41 | −1.54 | −0.22 | −3.908 | 0.005214 |
| cg02826078 | 16 | 34583045 | | 0.4 | 0.59 | 1.45 | 0.18 | 3.545 | 0.005886 |
| cg10331082 | 17 | 6796906 | ALOX12P2 | 0.6 | 0.42 | −1.44 | −0.18 | −3.891 | 0.00369 |
| cg20995835 | 17 | 18528589 | CCDC144B | 0.62 | 0.44 | −1.42 | −0.18 | −4.258 | 0.0055 |
| cg16434502 | 17 | 61434321 | TANC2 | 0.75 | 0.57 | −1.32 | −0.18 | −6.403 | 0.000575 |
| cg22874255 | 19 | 1768630 | ONECUT3 | 0.5 | 0.31 | −1.59 | −0.19 | −3.692 | 0.007847 |
| cg00249093 | 19 | 5804642 | | 0.47 | 0.32 | −1.48 | −0.15 | −3.236 | 0.009037 |
| cg16474696 | 19 | 13875014 | MRI1 | 0.14 | 0.37 | 2.72 | 0.23 | 3.653 | 0.007716 |
| cg25602603 | 19 | 22320744 | | 0.86 | 0.63 | −1.35 | −0.22 | −8.3 | 0.000018 |
| cg17716663 | 19 | 45978434 | FOSB | 0.25 | 0.45 | 1.78 | 0.2 | 4.384 | 0.003893 |
| cg18376773 | 20 | 44878374 | CDH22 | 0.26 | 0.49 | 1.86 | 0.23 | 4.948 | 0.000987 |
| cg20643029 | 21 | 37915044 | CLDN14 | 0.51 | 0.28 | −1.84 | −0.23 | −3.886 | 0.00428 |
| cg13007784 | 22 | 30901249 | SEC14L4 | 0.36 | 0.55 | 1.52 | 0.19 | 4.81 | 0.001476 |

90 probe sets (specific CpG dinucleotides listed above) satisfied the comparison filtering criteria

Results:

Determination of the percent DNA methylation of the DNA methylation biomarkers comprising one or more of the differentially methylated genes listed in Tables 1, 2, 3, or 4, infra was shown to be predictive of the response to the TKI/kinase inhibitor Rigosertib in patients with refractory myelodysplastic syndrome. These results were demonstrated in methylation heat map as depicted in FIG. 1, and in the validations by bisulfite DNA sequencing as depicted in FIGS. 2 and 3, respectively. The Infinium cross reference number, chromosome location, base pair position, gene name, baseline mean, experiment mean, fold change, difference of means, t statistic and p value for each of the specific loci of the DNA methylation profile genes are listed in Table 1, supra.

Conclusion:

In this Example, by applying DNA methylation profiling (Illumina 450K BeadChips) to bone marrow samples from a clinically well-characterized series of patients with myelodysplastic syndrome (MDS), the inventors surprisingly found that the methylation pattern of a small discrete panel of genes can effectively predict the presence of absence of a therapeutic response to a general kinase inhibitor drug, namely, Rigosertib, with validation of the BeadChip data results by gold-standard bisulfite sequencing.

Accordingly, the discrete panel of DNA methylation biomarkers listed in Table 1, or any specific sub-combination thereof may be used in a diagnostic method, wherein the extent of methylation of the DNA methylation profile marker in the subject sample's DNA region is predictive of the resistance or responsiveness of a broad specificity kinase inhibitor for treatment of a subject refractory cancer. Thus, this methylation profiling is able to distinguish between patients who are likely to be therapeutically resistant to or therapeutically responsive to Rigosertib.

Such advance notification of the therapeutic effectiveness of a kinase inhibitor in a patient with refractory MDS would be a valuable time saving and/or potentially life-saving diagnostic tool in the treatment of this debilitating cancer.

### Example 2

### Association of Hypermethylated Genes with Responder Predictive Loci DNA Methylation Signature Profile (Illumina 450K Beadchips) of MDS Bone Marrow Biopsy Samples is Predictive of Response to Rigosertib

Methods:

Pre-therapy bone marrow mononuclear cells from 32 patients were analyzed using the Illumina 450K methylation array platform.

Results:

After adding one more complete responder (CR) and 9 more non-responder (NR) patients, to the series of MDS patient pre-therapy bone marrow biopsy samples being analyzed with DNA methylation profiling (Illumina 450K Beadchips), and analyzing the methylation values in this expanded sample set, seventeen (17) of the marker loci from the original set [(sample numbers 1-17, respectively (highlighted in bold)] persist as predictive of response with individual (marker-by-marker) T-test p-values<0.01 and absolute differences in fractional methylation >0.10, as depicted in Table 2, infra. Additional potential marker loci [sample numbers 18-137, respectively (non-bolded)] are also detected in this expanded series, as depicted in Table 2, infra.

TABLE 2
Further predictive loci DNA methylation signature profile
(Illumina 450K Beadchips) of MDS bone marrow biopsy samples

| | | | | | T test p-value | CR minus | | |
|---|---|---|---|---|---|---|---|---|
| No. | Probe Set | Ch | Ch Position | Gene Symbol | CR vs. NR | NR¹ | CR² | NR³ |
| 1 | cg13153466 | 11 | 123008499 | ASAM | 5.45367E−05 | 0.25 | 0.62 | 0.38 |
| 2 | cg22874255 | 19 | 1768630 | ONECUT3 | 0.000194805 | 0.17 | 0.49 | 0.32 |
| 3 | cg13007784 | 22 | 30901249 | SEC14L4 | 0.000195728 | −0.20 | 0.35 | 0.55 |
| 4 | cg27518976 | 1 | 23886730 | ID3 | 0.000435321 | 0.20 | 0.58 | 0.38 |
| 5 | cg14116129 | 5 | 1140748 | | 0.000534232 | −0.13 | 0.28 | 0.41 |
| 6 | cg14454798 | 5 | 173314559 | CPEB4 | 0.001259969 | −0.19 | 0.48 | 0.66 |
| 7 | cg11489090 | 8 | 10405250 | | 0.001648967 | −0.19 | 0.32 | 0.51 |
| 8 | cg12252090 | 8 | 49833279 | SNAI2 | 0.00203496 | 0.16 | 0.57 | 0.41 |
| 9 | cg25976440 | 4 | 69239481 | | 0.002259781 | 0.18 | 0.42 | 0.24 |
| 10 | cg01564135 | 7 | 27281216 | EVX1 | 0.002380637 | −0.16 | 0.29 | 0.45 |
| 11 | cg20643029 | 21 | 37915044 | CLDN14 | 0.002422529 | 0.16 | 0.49 | 0.33 |
| 12 | cg09234936 | 2 | 3751890 | | 0.002654065 | −0.19 | 0.46 | 0.65 |
| 13 | cg25360385 | 12 | 51786547 | GALNT6 | 0.002747914 | 0.23 | 0.69 | 0.46 |
| 14 | cg15463966 | 3 | 148446998 | AGTR1 | 0.003588888 | 0.11 | 0.39 | 0.28 |
| 15 | cg16546703 | 5 | 149682795 | ARSI | 0.004092982 | −0.11 | 0.21 | 0.32 |
| 16 | cg13890649 | 9 | 79629843 | | 0.004225025 | 0.13 | 0.43 | 0.30 |
| 17 | cg15127250 | 12 | 51786489 | GALNT6 | 0.005392812 | 0.17 | 0.49 | 0.31 |
| 18 | cg11829608 | 1 | 207224549 | YOD1 | 1.36396E−05 | −0.19 | 0.21 | 0.40 |
| 19 | cg00585072 | 5 | 140186983 | PCDHA2 | 7.50247E−05 | 0.17 | 0.66 | 0.49 |
| 20 | cg04391972 | 4 | 182569038 | | 0.000166558 | −0.13 | 0.25 | 0.38 |
| 21 | cg18171855 | 10 | 2543474 | | 0.000221235 | 0.21 | 0.79 | 0.58 |
| 22 | cg09533869 | 8 | 97747124 | PGCP | 0.000269736 | 0.38 | 0.61 | 0.23 |
| 23 | cg20845050 | 8 | 146053666 | ZNF7 | 0.000334331 | 0.20 | 0.65 | 0.45 |
| 24 | cg03391002 | 12 | 108566377 | WSCD2 | 0.000348768 | −0.20 | 0.41 | 0.61 |
| 25 | cg22532079 | 3 | 100712058 | ABI3BP | 0.000386359 | 0.24 | 0.64 | 0.40 |
| 26 | cg12995113 | 11 | 123008408 | ASAM | 0.000395522 | 0.24 | 0.51 | 0.27 |
| 27 | cg17054386 | 2 | 219855054 | CRYBA2 | 0.000430962 | 0.16 | 0.60 | 0.44 |
| 28 | cg08482215 | 7 | 5111529 | LOC389458 | 0.000497427 | 0.19 | 0.71 | 0.53 |
| 29 | cg02793451 | 16 | 52582072 | TOX3 | 0.000565149 | 0.21 | 0.53 | 0.32 |
| 30 | cg15028548 | 3 | 100712322 | ABI3BP | 0.000565876 | 0.17 | 0.54 | 0.38 |
| 31 | cg22828989 | 10 | 124903224 | | 0.000630312 | −0.11 | 0.17 | 0.28 |
| 32 | cg22431184 | 17 | 37753528 | | 0.00063071 | −0.11 | 0.15 | 0.26 |
| 33 | cg14587335 | 17 | 77478600 | HRNBP3 | 0.000635665 | −0.10 | 0.21 | 0.31 |
| 34 | cg09309269 | 17 | 30770961 | PSMD11 | 0.000697839 | −0.19 | 0.19 | 0.38 |
| 35 | cg07589531 | 10 | 13970236 | FRMD4A | 0.000764451 | 0.29 | 0.87 | 0.58 |
| 36 | cg19592671 | 15 | 51369862 | TNFAIP8L3 | 0.000777908 | 0.20 | 0.51 | 0.31 |
| 37 | cg20191338 | 18 | 77905663 | LOC100130522 | 0.00083745 | 0.14 | 0.41 | 0.27 |
| 38 | cg07283849 | 7 | 97401062 | | 0.000902903 | 0.13 | 0.40 | 0.26 |
| 39 | cg16163419 | 3 | 100712326 | ABI3BP | 0.001103071 | 0.17 | 0.57 | 0.40 |
| 40 | cg16263180 | 5 | 140710509 | PCDHGA1 | 0.001112503 | 0.21 | 0.63 | 0.42 |
| 41 | cg03513464 | 1 | 58898672 | | 0.001127392 | 0.19 | 0.68 | 0.49 |
| 42 | cg13287964 | 18 | 77905751 | LOC100130522 | 0.001243984 | 0.16 | 0.52 | 0.36 |
| 43 | cg03105348 | 10 | 50599620 | DRGX | 0.001244576 | 0.18 | 0.56 | 0.38 |
| 44 | cg25529393 | 6 | 27858380 | HIST1H3J | 0.001394605 | −0.14 | 0.10 | 0.25 |
| 45 | cg10210594 | 1 | 208132787 | | 0.00140106 | −0.11 | 0.07 | 0.18 |
| 46 | cg06087349 | 1 | 214154085 | | 0.001487031 | −0.12 | 0.30 | 0.42 |
| 47 | cg12061113 | 18 | 77905747 | LOC100130522 | 0.001652405 | 0.17 | 0.48 | 0.31 |
| 48 | cg00916536 | 2 | 87017419 | CD8A | 0.001671785 | −0.12 | 0.19 | 0.31 |
| 49 | cg20971158 | 11 | 35159382 | CD44 | 0.001718896 | 0.16 | 0.61 | 0.45 |
| 50 | cg05204123 | 17 | 1613017 | TLCD2 | 0.001724185 | 0.11 | 0.33 | 0.22 |
| 51 | cg22510727 | 12 | 64541117 | SRGAP1 | 0.001811643 | 0.24 | 0.65 | 0.40 |
| 52 | cg17774559 | 5 | 1879698 | IRX4 | 0.001892769 | −0.18 | 0.25 | 0.43 |
| 53 | cg23136738 | 11 | 925521 | AP2A2 | 0.002047752 | −0.17 | 0.32 | 0.49 |
| 54 | cg04863005 | 1 | 59043208 | TACSTD2 | 0.002053025 | 0.26 | 0.82 | 0.56 |
| 55 | cg05406088 | 15 | 66947617 | | 0.002062761 | −0.20 | 0.14 | 0.34 |
| 56 | cg02162534 | 10 | 133956875 | JAKMIP3 | 0.002204347 | −0.14 | 0.22 | 0.36 |
| 57 | cg02532022 | 15 | 91566346 | VPS33B | 0.002258491 | −0.13 | 0.32 | 0.45 |
| 58 | cg10653240 | 1 | 51810892 | TTC39A | 0.002274106 | −0.11 | 0.12 | 0.22 |
| 59 | cg20302533 | 7 | 39170763 | POU6F2 | 0.002298735 | −0.21 | 0.41 | 0.62 |
| 60 | cg24377133 | 8 | 144170375 | | 0.002414702 | 0.11 | 0.30 | 0.19 |
| 61 | cg04747322 | 5 | 121647308 | SNCAIP | 0.002420224 | 0.17 | 0.50 | 0.33 |
| 62 | cg05767421 | 8 | 120221185 | MAL2 | 0.002486536 | 0.15 | 0.46 | 0.31 |
| 63 | cg05273302 | 19 | 2095560 | C19orf36 | 0.002514263 | −0.16 | 0.29 | 0.45 |
| 64 | cg11723923 | 13 | 112820997 | | 0.002582507 | 0.27 | 0.86 | 0.58 |
| 65 | cg18850127 | 7 | 39170497 | POU6F2 | 0.002610818 | −0.25 | 0.39 | 0.64 |
| 66 | cg06092953 | 18 | 77905699 | LOC100130522 | 0.002720686 | 0.14 | 0.39 | 0.25 |
| 67 | cg10089193 | 16 | 1140939 | C1QTNF8 | 0.002870867 | 0.15 | 0.47 | 0.32 |
| 68 | cg19774868 | 18 | 77905408 | LOC100130522 | 0.002926848 | 0.14 | 0.43 | 0.29 |
| 69 | cg18393958 | 11 | 82443149 | FAM181B | 0.003206187 | −0.13 | 0.14 | 0.27 |
| 70 | cg23026864 | 5 | 140306231 | PCDHA7 | 0.003214138 | −0.13 | 0.17 | 0.30 |
| 71 | cg24353443 | 3 | 73674166 | PDZRN3 | 0.003280348 | −0.16 | 0.21 | 0.37 |
| 72 | cg20247486 | 1 | 119544329 | | 0.003373747 | 0.15 | 0.47 | 0.32 |
| 73 | cg09993319 | 10 | 131529435 | MGMT | 0.003470192 | 0.33 | 0.72 | 0.40 |
| 74 | cg12854458 | 6 | 46703670 | PLA2G7 | 0.003488651 | 0.14 | 0.46 | 0.32 |
| 75 | cg20954533 | 5 | 71462729 | MAP1B | 0.003524395 | −0.10 | 0.23 | 0.33 |
| 76 | cg18963953 | 6 | 99288299 | | 0.003578099 | −0.14 | 0.28 | 0.42 |
| 77 | cg16376902 | 5 | 140612229 | | 0.00369598 | −0.13 | 0.22 | 0.35 |
| 78 | cg10831607 | 12 | 115134374 | | 0.003750995 | −0.14 | 0.10 | 0.24 |
| 79 | cg15903915 | 7 | 31381065 | NEUROD6 | 0.003845476 | 0.16 | 0.47 | 0.31 |
| 80 | cg06738356 | 14 | 24046374 | JPH4 | 0.003848081 | −0.13 | 0.07 | 0.21 |
| 81 | cg06508320 | 3 | 179660149 | PEX5L | 0.003851905 | −0.12 | 0.16 | 0.28 |
| 82 | cg26527775 | 12 | 133758599 | ZNF268 | 0.003965149 | −0.11 | 0.10 | 0.20 |
| 83 | cg18288462 | 10 | 103986268 | ELOVL3 | 0.004019663 | 0.14 | 0.39 | 0.26 |
| 84 | cg26621770 | 12 | 115131447 | | 0.004310209 | 0.17 | 0.48 | 0.30 |
| 85 | cg09939079 | 12 | 69753248 | YEATS4 | 0.004314848 | −0.10 | 0.22 | 0.33 |
| 86 | cg14468236 | 4 | 3746931 | | 0.004386141 | 0.20 | 0.62 | 0.43 |
| 87 | cg14983172 | 5 | 73244708 | | 0.004451492 | 0.25 | 0.67 | 0.42 |
| 88 | cg12078775 | 6 | 30419543 | | 0.004527954 | −0.18 | 0.16 | 0.34 |
| 89 | cg15425811 | 22 | 31318181 | C22orf27 | 0.004529984 | 0.16 | 0.41 | 0.25 |
| 90 | cg16699148 | 1 | 59043255 | TACSTD2 | 0.004565697 | 0.22 | 0.68 | 0.46 |
| 91 | cg00058329 | 12 | 133195094 | P2RX2 | 0.004619739 | −0.10 | 0.10 | 0.21 |
| 92 | cg18255240 | 15 | 27111993 | GABRA5 | 0.004639908 | −0.10 | 0.23 | 0.33 |
| 93 | cg17397150 | 6 | 101840567 | | 0.004641596 | −0.12 | 0.18 | 0.30 |
| 94 | cg27572120 | 6 | 30419551 | | 0.004815149 | −0.12 | 0.26 | 0.38 |
| 95 | cg02010738 | 19 | 2095466 | C19orf36 | 0.005228152 | −0.14 | 0.14 | 0.28 |
| 96 | cg26203879 | 10 | 120968844 | GRK5 | 0.005350105 | −0.11 | 0.12 | 0.22 |
| 97 | cg27370471 | 15 | 101932559 | PCSK6 | 0.005609408 | −0.11 | 0.22 | 0.33 |
| 98 | cg26667586 | 7 | 23636775 | CCDC126 | 0.005615907 | −0.13 | 0.18 | 0.31 |
| 99 | cg14204586 | 1 | 155931858 | ARHGEF2 | 0.005757675 | −0.11 | 0.19 | 0.31 |
| 100 | cg07635227 | 16 | 4714815 | MGRN1 | 0.005872086 | −0.12 | 0.18 | 0.30 |
| 101 | cg17858192 | 4 | 16077807 | PROM1 | 0.005895354 | 0.22 | 0.40 | 0.19 |
| 102 | cg02627966 | 7 | 54899537 | | 0.005921868 | −0.15 | 0.24 | 0.39 |
| 103 | cg08409113 | 17 | 40937365 | WNK4 | 0.005929761 | −0.12 | 0.08 | 0.21 |
| 104 | cg06596654 | 16 | 86527923 | | 0.005936314 | −0.12 | 0.13 | 0.26 |
| 105 | cg13371839 | 19 | 22605151 | ZNF98 | 0.005995058 | −0.11 | 0.13 | 0.25 |
| 106 | cg00688297 | 8 | 145752292 | LRRC24 | 0.006290125 | −0.14 | 0.17 | 0.30 |
| 107 | cg13428978 | 8 | 1993987 | MYOM2 | 0.006310188 | −0.19 | 0.36 | 0.55 |
| 108 | cg22728782 | 16 | 88937386 | | 0.006314191 | −0.14 | 0.25 | 0.39 |
| 109 | cg05640128 | 7 | 154002270 | DPP6 | 0.006447219 | −0.11 | 0.21 | 0.32 |
| 110 | cg24284460 | 3 | 42139507 | TRAK1 | 0.006855178 | −0.13 | 0.10 | 0.23 |
| 111 | cg05364038 | 16 | 5500503 | | 0.006869412 | −0.13 | 0.07 | 0.20 |
| 112 | cg20005518 | 17 | 33759484 | SLFN12 | 0.006880956 | −0.21 | 0.10 | 0.32 |
| 113 | cg21093807 | 3 | 56717625 | C3orf63 | 0.00700443 | −0.16 | 0.31 | 0.47 |
| 114 | cg09399371 | 14 | 24702584 | GMPR2 | 0.007206278 | −0.10 | 0.19 | 0.30 |
| 115 | cg03736795 | 4 | 174444307 | | 0.007328753 | −0.12 | 0.09 | 0.21 |
| 116 | cg01483139 | 4 | 187549458 | FAT1 | 0.00764693 | −0.19 | 0.15 | 0.34 |
| 117 | cg24502342 | 20 | 37359533 | | 0.007683811 | 0.17 | 0.40 | 0.23 |
| 118 | cg06178563 | 20 | 21494712 | NKX2-2 | 0.007718035 | −0.13 | 0.15 | 0.28 |
| 119 | cg24407607 | 6 | 116753994 | DSE | 0.007895758 | 0.28 | 0.70 | 0.42 |
| 120 | cg24502334 | 1 | 243264842 | LOC731275 | 0.008075732 | −0.10 | 0.20 | 0.30 |
| 121 | cg02705835 | 3 | 49203908 | CCDC71 | 0.008234847 | −0.13 | 0.15 | 0.28 |
| 122 | cg20381115 | 15 | 45671148 | LOC145663 | 0.008266148 | −0.14 | 0.18 | 0.32 |
| 123 | cg08149193 | 11 | 44333067 | ALX4 | 0.008354195 | −0.13 | 0.13 | 0.26 |
| 124 | cg01938018 | 1 | 179545425 | NPHS2 | 0.008372163 | −0.12 | 0.19 | 0.31 |
| 125 | cg26871372 | 4 | 41867517 | | 0.008476659 | −0.10 | 0.14 | 0.24 |
| 126 | cg22203547 | 5 | 145717010 | | 0.008571509 | −0.10 | 0.18 | 0.28 |
| 127 | cg06663305 | 17 | 8095813 | | 0.008651792 | −0.13 | 0.18 | 0.31 |
| 128 | cg09827761 | 15 | 66947392 | | 0.008759755 | −0.22 | 0.10 | 0.32 |
| 129 | cg18252903 | 3 | 147072643 | | 0.00923771 | −0.14 | 0.20 | 0.34 |
| 130 | cg16013006 | 19 | 55790967 | HSPBP1 | 0.009297493 | −0.11 | 0.19 | 0.30 |
| 131 | cg08387835 | 8 | 1094502 | | 0.009424812 | −0.15 | 0.21 | 0.36 |
| 132 | cg08530838 | 5 | 114193858 | | 0.009448805 | −0.16 | 0.13 | 0.29 |
| 133 | cg26475168 | 19 | 49016629 | LMTK3 | 0.00947928 | 0.15 | 0.41 | 0.26 |
| 134 | cg15932284 | 11 | 126153561 | TIRAP | 0.009737213 | −0.15 | 0.20 | 0.35 |
| 135 | cg23439460 | 10 | 102809954 | | 0.009757756 | 0.18 | 0.46 | 0.28 |
| 136 | cg20354552 | 17 | 33760249 | SLFN12 | 0.009809835 | −0.13 | 0.07 | 0.20 |

¹Difference in the methylation status [CR minus NR].
²Average methylation status of Complete Responder (CR).
³Average methylation status of Non-Responder (NR).

In summary, seven (7) of 32 MDS patients had complete response or CR (Transfusion independence (TI)+increase in hemoglobin (Hb)>2 g/dL), ten (10) had partial response or PR (TI without Hb increase) and fifteen (15) had no response or NR. Supervised hierarchical clustering by methylation intensity demonstrated a distinct profile associated with complete responders. Bisulfite sequencing (which allows quantification of multiple consecutive CpGs in an amplicon) of several differentially methylated loci confirmed the Illumina 450K data.

Table 3 infra provides the sequence contexts (SEQ ID NOS. 1-17, respectively) for the CpG dinucleotides that persisted as strongly predictive of drug response in the expanded series of MDS cases treated with Rigosertib as indicated in bold font in Table 2 supra. The predictive CpG is indicated as [CG] in each sequence of SEQ ID NOS. 1-17, respectively. The methylation status of these CpGs can be scored by Illumina 450K BeadChips, according to the protocol of the manufacturer, or by related methods including standard bisulfite sequencing or methylation-sensitive pyro sequencing.

TABLE 3
Sequence contexts for CpG dinucleotides for MDS cases treated with
Rigosertib

| | | Chr | | |
|---|---|---|---|---|
| | | Position | | |
| | | Human | | |
| | | Genome | Gene | |
| probe set | Chr | Build 37 | Symbol | Sequence context of predictive CpG [CG] |
| cg13153466 | 11 | 123008499 | ASAM | TCGCAAACAAAGAAGTCAGTTGGGCGCCCCGAAGC |
| | | | | TGCTGGC |
| | | | | CTTCTGAGTGGCGGAAGG[CG]CCAGGCTCGGTGAT |
| | | | | TGAAACC |
| | | | | ACATGTGCTGGAGGTTTCAAGAGCAGCGACCCAGA |
| | | | | CACC (SEQ. ID No. 1) |
| cg22874255 | 19 | 1768630 | ONECUT3 | CGCCAGCTTGGTGGCCAAGGTCTGTGTTGGGGGCC |
| | | | | ACTCTCTA |
| | | | | TCCCCCCGACTACAGGT[CG]GGAGCTGCTGGTGAT |
| | | | | GGAACCA |
| | | | | GGGGCTGAGTATGCAGTGCTGGCCACATACTAGGA |
| | | | | GAA (SEQ. ID No. 2) |
| cg13007784 | 22 | 30901249 | SEC14L4 | AACAAATGTTGCAACCTGGACATGAAAAGCAGTCT |
| | | | | GTCTACCC |
| | | | | AAGGCTGCCCCCGCAGC[CG]GATGGCAGCGCCACT |
| | | | | CCCTCCCC |
| | | | | CACAACCGGGGAACAAGGACCAGGGCCGGGTGGT |
| | | | | GGA (SEQ. ID No. 3) |
| cg27518976 | 1 | 23886730 | ID3 | CCCATGCTTTTTGCATGGGGAAAAAAGGAGCCTGG |
| | | | | ACCCTCTG |
| | | | | CCCCATAAACTCCATAA[CG]AACTAAATATATGCAC |
| | | | | AGTTGTG |
| | | | | CTTTGTGAGTTCAATTAGAATTTTGGACAAGGTTTT |
| | | | | A (SEQ. ID No. 4) |
| cg14116129 | 5 | 1140748 | | TGGGGTTCAGGACGGTGCCAGCATGCCCAGCAATC |
| | | | | CCGGAGT |
| | | | | TCCCCGTGCTCAGGAACT[CG]CACCGCTGGCTCCAC |
| | | | | GCTCTCT |
| | | | | ACTCCAGTGGGACAAGAAAGCTCTGCTCACCAGGC |
| | | | | AAC (SEQ. ID No. 5) |
| cg14454798 | 5 | 173314559 | CPEB4 | AATACCAGACTAACAAGTTTGGCCTTTATCTTCGAA |
| | | | | ATAATGG |
| | | | | GCAACCATAGAAGAAAT[CG]GAGCATGTGAGGGT |
| | | | | CAAGATAA |
| | | | | AATTTTAGTTTTGGAATTTTCCCTCTGGTATTGCGTG |
| | | | | T (SEQ. ID No. 6) |
| cg11489090 | 8 | 10405250 | | TTTCCTCCTTTTCTTTCCTTCCCTTCCTTCCTCCCTCTC |
| | | | | CCCTTCCC |
| | | | | CCACCCCCCGCCC[CG]CCCCGCCCCAGTCTTGGTGG |
| | | | | CTTGTTCC |
| | | | | GGATCTGGTGTTGCTCATTCACTCATCAAACA |
| | | | | (SEQ. ID No. 7) |
| cg12252090 | 8 | 49833279 | SNA12 | AGCTGCACGGAGCTATAGGTGCTCTGAAGTCAGAC |
| | | | | AGTGCAG |
| | | | | CCAAGCCAGTGCCTTCGC[CG]TCCCCATTGAGGAA |
| | | | | GGAGAGC |
| | | | | CTCAACATTATTTTTAAACATACAGAAAAGTTGTTTT |
| | | | | CC (SEQ. ID No. 8) |
| cg25976440 | 4 | 69239481 | | TGCCATTGGCAGAACAAAGCAGTCTCGGCCTGGCT |
| | | | | TGGCAGC |
| | | | | ATCCAAAAGGACCGCAGC[CG]CCCTGCCGCTGCAC |
| | | | | ACGGTGC |
| | | | | ATCTGATTGGCGGAACCCAAACCCTTCCGCAGCCCT |
| | | | | CCC (SEQ. ID No. 9) |
| cg01564135 | 7 | 27281216 | EVX1 | GTCCCTACTTACTTGCTGTTTTAGGGCATTTTCAGC |
| | | | | GACTTCACT |
| | | | | CTCTTCTACCTCAAA[CG]CCATCCACTTTTGGAGAC |
| | | | | AGGTACCA |
| | | | | CTGCCCCTAGGCAGGGACTTGGGAGAGGACCTTA |
| | | | | (SEQ. ID No. 10) |
| cg20643029 | 21 | 37915044 | CLDN14 | TCCCAGAGGCCCCCCAGCAGCCCCCGAGCAGTCCC |
| | | | | CGGAGCG |
| | | | | CTGGGGGACTCTGCAGCG[CG]CAGGTGACCACGC |
| | | | | AGAGACAG |
| | | | | CCCCAGCGCGATGAATAATTGAGGCGCAGAGTGG |
| | | | | AATTA (SEQ. ID No. 11) |
| cg09234936 | 2 | 3751890 | | CCGCCCCCAGGGTCCCTTCCTATGGGGCAGGGCGG |
| | | | | GCCCTGG |
| | | | | CCGGCAGCGATTCAGGAC[CG]CCCCAGAGACGGC |
| | | | | CAGGAGG |
| | | | | GCGTGGGGGAGGGGCGTGGGGCAGAAACAGGGG |
| | | | | TGTGGAG (SEQ. ID No. 12) |
| cg25360385 | 12 | 51786547 | GALNT6 | CTCGACCTTGAGAACCCTGAACAATTAGTGGCAGG |
| | | | | GCTGGAAT |
| | | | | GAGAATCCTGGTAAACC[CG]CCTCCTAAGCCGGTG |
| | | | | TTCGGAAT |
| | | | | TACCCCGCCAGGGCCCGTGCTGCTCAGTGGTCCTCT |
| | | | | C (SEQ. ID No. 13) |
| cg15463966 | 3 | 148446998 | AGTR1 | TTGCTGGTAATAAAAGCAGAAGTCACGTGCTGAAC |
| | | | | GTGAAGG |
| | | | | TGAGCAGAGGAGAACTTG[CG]ATGGCAAAGTTAA |
| | | | | AAACAAGA |
| | | | | GGAGATGATGGTCTTGGTGTGGCACAGGATGTTAA |
| | | | | AAAA (SEQ. ID No. 14) |
| cg16546703 | 5 | 149682795 | ARS1 | CTAGTCCAACCGGGTCATTCTACAGATGGAGAAAC |
| | | | | TGAGGCCC |
| | | | | GGAGATGAGCCTCAACG[CG]TCTAAGTCACAGAAC |
| | | | | AAGTCAG |
| | | | | TGGCTATGTCTCTAAGGCTCACCGTGGCTGTTGGA |
| | | | | GTG (SEQ. ID No. 15) |
| cg13890649 | 9 | 79629843 | | CTCCACCCTCGGGCATTCCGAAAAGTCAAATCGAG |
| | | | | CTTATGCA |
| | | | | CTCAAATCCAAGTAGGG[CG]GAGCCAAACCTGTGT |
| | | | | CCTGGGC |
| | | | | GCCTGCCGGGCAAGGCGCGGGGACTGGACGGGTC |
| | | | | GGAA (SEQ. ID No. 16) |
| cg15127250 | 12 | 51786489 | GALNT6 | ACTCGGGTTGGGCCCGGCGACTTGTGTTTTAAAAG |
| | | | | CCCTCCGG |
| | | | | GTGATTTTGTGCATGCT[CG]ACCTTGAGAACCCTGA |
| | | | | ACAATTA |
| | | | | GTGGCAGGGCTGGAATGAGAATCCTGGTAAACCC |
| | | | | GCC (SEQ. ID No. 17) |

| | T test p- | Diff in | | |
|---|---|---|---|---|
| | value | methylation on | CR AVG | NR AVG |
| probe set | CR vs NR | CR minus NR | Methylation | methylation |
| cg13153466 | 5.45367E-05 | 0.25 | 0.62 | 0.38 |
| cg22874255 | 0.000194805 | 0.17 | 0.49 | 0.32 |
| cg13007784 | 0.000195728 | -.20 | 0.35 | 0.55 |
| cg27518976 | 0.000435321 | .20 | 0.58 | 0.38 |
| cg14116129 | 0.000534232 | -0.13 | 0.28 | 0.41 |
| cg14454798 | 0.001259969 | -0.19 | 0.48 | 0.66 |
| cg11489090 | 0.001648967 | -0.19 | 0.32 | 0.51 |
| cg12252090 | 0.00203496 | 0.16 | 0.57 | 0.41 |
| cg25976440 | 0.002259781 | 0.18 | 0.42 | 0.24 |
| cg01564135 | 0.002380637 | -0.16 | 0.29 | 0.45 |
| cg20643029 | 0.002422529 | 0.16 | 0.49 | 0.33 |
| cg09234936 | 0.002654065 | -0.19 | 0.46 | 0.65 |
| cg25360385 | 0.002747914 | 0.23 | 0.69 | 0.46 |
| cg15463966 | 0.003588888 | 0.11 | 0.39 | 0.28 |
| cg16546703 | 0.004092982 | -0.11 | 0.21 | 0.32 |
| cg13890649 | 0.004225025 | 0.13 | 0.43 | 0.30 |
| cg15127250 | 0.005392812 | 0.17 | 0.49 | 0.31 |

Table 4 infra provides the sequence contexts for the CpG dinucleotides for the CASZ1 and FOSB responder and non-responder predictive loci DNA methylation signature profiles (SEQ ID NOS. 18-20, respectively).

TABLE 4
Sequence contexts for the CpG dinucleotides for the CASZ1 and FOSB
responder and non-responder predictive loci DNA methylation signature profiles

| | | | | | Non- | |
|---|---|---|---|---|---|---|
| | | | | Responder | Responder | |
| | | | | mean (1^{st} | Mean (1st | |
| | | | | series of | series of | |
| | | Base | Gene | MDS | MDS | Fold |
| probe set | chr | Position | Name | cases) | cases) | Change |
| cg16101008 | 1 | 10698719 | CASZ1 | 0.58 | 0.39 | -1.51 |
| cg02396224 | 1 | 10698794 | CASZ1 | 0.44 | 0.24 | -1.86 |
| cg17716663 | 19 | 45978434 | FOSB | 0.25 | 0.45 | 1.78 |

| | | | | Sequence Context: the |
|---|---|---|---|---|
| | Diff of | t- | | predictive CpG dinucleotide |
| probe set | Means | Statistic | P value | is indicated by [CG] |
| cg16101008 | -0.2 | -0.37 | 0.004281 | CCGCGGGGAGGGGCGCCGGGGCAGCGGCCG |
| | | | | TGGAGCG[CG]CCGCATT |
| | | | | CGCCCGGGACCCCTGCTCCTAGGTTCCCTA |
| | | | | (SEQ. ID No. 18) |
| cg02396224 | -0.2 | -0.3457 | 0.00932 | CCCCGGGACAAGAGCACCTTCTGTTTCCCTGA |
| | | | | AGAGACC[CG]GCCTCCC |
| | | | | TAGAGCAGGGCCACCGCCGCCGCCTGTGT |
| | | | | (SEQ. ID No. 19) |
| cg17716663 | 0.2 | 4.384 | 0.003893 | CTTGGTTCTGCACTGTTGCCAATAAAAAGCTC |
| | | | | TTAAAAA[CG]CATTCGCC |
| | | | | AGGCTACAGTGTTTATTTCCTCCTAACAC |
| | | | | (SEQ. ID No. 20) |

In general, hypermethylation of a group of genes was associated with responders. Functional annotation of the hypo and hypermethylated genes which best distinguished CRs from NRs showed that the genes most affected by methylation were related to regulation of transcription followed by genes involved in cell-cell adhesion, inflammatory response, apoptosis and proliferation.

In this analysis, the observed correlation of hematological response to genomic methylation status suggested the possibility of preselecting transfusion dependent lower risk MDS patients likely to benefit from treatment with rigosertib.

Conclusion:

Other than the exemplary diagnostic and prognostic utilities described herein, additional possible uses include application of this panel of DNA methylation biomarkers in a state-of-the-art personalized medicine approach to treating MDS.

The entire disclosure of each document cited (including patents, patent applications, journal articles, abstracts, laboratory manuals, books, or other disclosures) in the Background of the Invention, Detailed Description, and Examples is hereby incorporated herein by reference.

The foregoing description of some specific embodiments provides sufficient information that others can, by applying current knowledge, readily modify or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. In the drawings and the description, there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein. Those skilled in the art will recognize, or be able to ascertain using no more than routine experimentation, many equivalents to the embodiments of the invention described herein. Such equivalents are encompassed by the following claims.

What is claimed is:

1. A diagnostic method for predicting the therapeutic efficacy of a broad specificity kinase inhibitor in a subject with refractory cancer comprising: (a) obtaining a test genomic DNA sample from a tissue of the subject; (b) analyzing the DNA methylation profile of the test genomic DNA sample; and (c) comparing the DNA methylation profile of the one or more CpG dinucleotide sequences of the test genomic DNA sample with corresponding sequences of a discrete panel of DNA methylation profile biomarkers comprising one or more genes of the differentially methylated genes listed in Tables 1, 2, 3, or 4, or any sub-combination thereof to determine the locus specific DNA methylation profile pattern of the test genomic DNA sample in order to predict the therapeutic efficacy of a broad specificity kinase inhibitor for treatment of a subject with refractory cancer.

2. The method of claim 1, wherein the tissue of the subject is derived from a cancer tissue, or a putative cancer tissue.

3. The method of claim 1, wherein the refractory cancer is a hematological cancer.

4. The method of claim 3, wherein the refractory hematological cancer is myelodysplastic syndrome.

5. The method of claim 1, wherein the test genomic DNA sample is derived from bone marrow of myelodysplastic syndrome patients.

6. The method of claim 5, wherein the discrete panel of DNA methylation profile biomarkers comprises a DNA methylation profile of genes comprising one or more of the differentially methylated genes listed in Tables 1, 2, 3, or 4, any variants thereof, or any sub-combination thereof.

7. The method of claim 1, wherein the broad specificity kinase inhibitor comprises a PT 3-kinase, a polo-like kinase 1 (PLK-1), or both.

8. The method of claim 1, wherein the broad specificity kinase inhibitor comprises a pharmaceutical composition comprising at least one compound of Formula 1 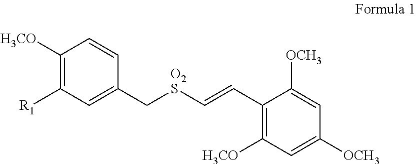
Where R₁ is selected from the group consisting of —NH₂, —NH—CH₂—COOH, —NH—CH(CH₃)—COOH, —NH—C(CH₃)2-COOH, —NH—CH₂—CH₂—OH and —N—(CH₂CH₂OH)₂ a pharmaceutically acceptable salt of such a compound, an anticancer agent, or a combination thereof.

9. The method of claim 1, wherein the broad specificity kinase inhibitor is Rigosertib.

10. The method of claim 1, wherein the refractory cancer comprises hematopoietic cancer, pancreatic cancer, head and neck cancer, cutaneous tumors, acute lymphoblastic leukemia (ALL), minimal residual disease (MRD) in acute lymphoblastic leukemia (ALL), acute myeloid leukemia (AML), lung cancer, breast cancer, ovarian cancer, prostate cancer, colon cancer, melanoma or other hematological cancer and solid tumors.

11. The method of claim 10, wherein the hematopoietic cancer is myelodysplastic syndrome.

12. The method of claim 1, wherein the determination of locus specific DNA methylation profile comprises a method selected from the group consisting of DNA sequencing using bisulfite treatment, restriction landmark genomic scanning, methylation-sensitive arbitrarily primed PCR, Southern analysis using a methylation-sensitive restriction enzyme, methylation-specific PCR, restriction enzyme digestion of PCR products amplified from bisulfite-converted DNA, and combinations thereof.

13. The method of claim 1, where the determination of locus specific DNA methylation profile comprises: (a) reacting the test genomic DNA sample with sodium bisulfite to convert unmethylated cytosine residues to uracil residues while leaving any 5-methylcytosine residues unchanged to create an exposed bisulfite-converted DNA sample having binding sites for primers specific for the bisulfite-converted DNA sample; (b) performing a PCR amplification procedure using top strand or bottom strand specific primers; (c) isolating the PCR amplification products; (d) performing a primer extension reaction using the gene specific primers to one or more of the differentially methylated genes listed in Tables 1, 2, 3, or 4, dNTPs and Taq polymerase, wherein the gene specific primer comprises from about a 15-mer to about a 22-mer length sequence that is complementary to the bisulfite-converted DNA sample and terminates immediately 5′ of the cytosine residue of the one or more CpG dinucleotide sequences; and (e) determining the locus specific DNA methylation profile of the one or more CpG dinucleotide sequences by determining the identity of the first primer-extended base against a discrete panel of DNA methylation biomarkers, wherein the locus-specific DNA methylation profile predicts the therapeutic efficacy of a broad specificity kinase inhibitor for treatment of a subject with refractory cancer.

14. The method of claim 13, wherein the dNTPs are labeled with a label selected from the group consisting of radiolabels and fluorescent labels, and wherein determining the identity of the first primer-extended base is by measuring incorporation of the labeled dNTPs.

15. The method of claim 1, wherein the test genomic DNA sample is derived from whole blood, buffy coat, isolated mononuclear cells, plasma, serum, bone marrow, stool, colonic effluent, urine, saliva, or a combination thereof.

16. The method of claim 1, wherein the refractory cancer comprises myelodysplastic syndrome, and the at least one DNA methylation profile biomarker is selected from the group consisting of RERE, CASZ1, KIAA1026, ID3, ADCY10, RNASEL, PGBD5, AKT3, SLC8Al, PLEKHH2, SGPP2, GNAT1, ALDH1L1, AGTR1, MSX1, KCNIP4, G3BP2, FLJ44606, PCDHA1, PCDHGA4, ARSI, CPEB4, SCAND3, BAT2, HLA-DRB1, MOCS1, SPACA1, LOC389458, EVX1, WNT16, SNAI2, HEY1, CRTAC1, HCCA2, C11orf58, AHNAK, ASAM, GALNT6, GALNT9, FLT1, DZIP1, ALOX12P2, CCDC144B, TANC2, ONECUT3, MRI1, FOSB, CDH22, CLDN14, and SEC14L4, any variants thereof, or any combination thereof.

17. The method of claim 1, wherein the refractory cancer comprises myelodysplastic syndrome, and the at least one DNA methylation profile biomarker is a FOSB gene or variant thereof that is hyper-methylated in non-responders.

18. The method of claim 1, wherein the refractory cancer comprises myelodysplastic syndrome, and the at least one DNA methylation profile biomarker is a CASZI gene or variant thereof that is hyper-methylated in responders.

19. The method of claim 1, wherein the determination of the locus specific DNA methylation profile is prior to, concomitant with, or subsequent to the administration of the compound of Formula 1.

20. The method of claim 1, wherein the method achieves diagnosis, prognosis, or treatment of the refractory cancer, or a combination thereof.

21. A method for treating a subject with refractory cancer comprising: (a) obtaining a test genomic DNA sample from a tissue of the subject; (b) analyzing the DNA methylation profile of the test genomic DNA sample; (c) comparing the DNA methylation profile of the one or more CpG dinucleotide sequences of the test genomic DNA sample with a corresponding sequences of a discrete panel of DNA methylation profile biomarkers comprising one or more of the differentially methylated genes listed in Tables 1, 2, 3, or 4, or any sub-combination thereof to determine a locus specific DNA methylation profile of the test genomic DNA sample to predict the therapeutic efficacy of a broad specificity kinase inhibitor for treatment of the subject with refractory cancer in advance; and (d) treating the subject with the broad specificity kinase inhibitor.

22. A test kit for predicting the therapeutic efficacy of a broad specificity kinase inhibitor in a subject with refractory cancer comprising ingredients and assays for determining the DNA methylation profile of a test genomic DNA sample and comparing the DNA methylation profile of the one or more CpG dinucleotide sequences of the test genomic DNA sample with the corresponding sequences of a discrete panel of DNA methylation biomarkers comprising one or more of the differentially methylated genes listed Tables 1, 2, 3, or 4, or any sub-combination thereof, wherein the locus-specific DNA methylation profile predicts the therapeutic efficacy of a broad specificity kinase inhibitor for treatment of a subject with refractory cancer.
